SEQUENCE LISTING (23 sequences)

SEQ ID NO: 1 - DNA, 11 bp, Artificial Sequence
  Synthetic
atcatctaagc

SEQ ID NO: 2 - DNA, 20 bp, Artificial sequence
  Synthetic
accgtgtgtttgggtgtgta

SEQ ID NO: 3 - DNA, 50 bp, Artificial sequence
  Synthetic
gagtacccatggcaatgaggagtacccatggcgatgaggacccatggcaa

SEQ ID NO: 4 - DNA, 52 bp, Artificial sequence
  Synthetic
gaggaccgcatggcgatgagtacccattgcgatgaggagttacccatggcaa

SEQ ID NO: 5 - DNA, 78 bp, Artificial sequence
  Synthetic
accgtgtgtttgggtgtgtatatgtatgcatgtttgtatttttgtgtctgtgtatatttgtgtgtttttgtctggcca

SEQ ID NO: 6 - DNA, 69 bp, Artificial sequence
  Synthetic
tgtgtttgtttggtgtgtatatgtatgcatgtatttttgtgtctgtgtagatttgtgtgtttttgtctg

SEQ ID NO: 7 - DNA, 71 bp, Artificial sequence
  Synthetic
tgtgtttgtttggtgtgtatatatgtatgcatgtatttttgtgtctgtgtagatttgtgtgtttttgtctg

SEQ ID NO: 8 - DNA, 60 bp, Artificial sequence
  Synthetic
tttgtttgatgtgtatatgtatgcatgtatttttgtgtctgtgtagatttgtgtgttttt

SEQ ID NO: 9 - DNA, 55 bp, Artificial sequence
  Synthetic
tttgttcggtgtgtatatgtatgcatgtagtgtctgtgtagatttgtgtgttttt

SEQ ID NO: 10 - DNA, 74 bp, Artificial sequence
  Synthetic
cagatcacgaggtcaagagattgagaccatccaggccaatgtggtgaaaccccatctctactaaaaatataaaa

SEQ ID NO: 11 - DNA, 66 bp, Artificial sequence
  Synthetic
acgaggtcaagaacgattgagaccatccaggccaatgcggtgaaaccccatctctactaaaaatat

SEQ ID NO: 12 - DNA, 74 bp, Artificial sequence
  Synthetic
  misc_feature (7)..(70): n is a, c, g, or t
cagatcnnnnnnnnnnnnnnnnnnnnnnnnnnnnnnnnnnnnnnnnnnnnnnnnnnnnnnnnnnnnnnnnaaaa

SEQ ID NO: 13 - DNA, 47 bp, Artificial sequence
  Synthetic
cagatcacgaggtcaagagattgagaccatccaggccaatgtggtga

SEQ ID NO: 14 - DNA, 20 bp, Artificial sequence
  Synthetic
cagatcacgaggtcaagaga

SEQ ID NO: 15 - DNA, 20 bp, Artificial sequence
  Synthetic
cagatcacgaggtcaggaga

SEQ ID NO: 16 - DNA, 20 bp, Artificial sequence
  Synthetic
cagatcacgagttcaagaga

SEQ ID NO: 17 - DNA, 20 bp, Artificial sequence
  Synthetic
cagatcacgagttcaggaga

SEQ ID NO: 18 - DNA, 20 bp, Artificial sequence
  Synthetic
ggtcaagagattgagaccat

SEQ ID NO: 19 - DNA, 20 bp, Artificial sequence
  Synthetic
ggtcaggagattgagaccat

SEQ ID NO: 20 - DNA, 20 bp, Artificial sequence
  Synthetic
gttcaagagattgagaccat

SEQ ID NO: 21 - DNA, 20 bp, Artificial sequence
  Synthetic
gttcaggagattgagaccat

SEQ ID NO: 22 - DNA, 20 bp, Artificial sequence
  Synthetic
ttgagaccatccaggccaat

SEQ ID NO: 23 - DNA, 20 bp, Artificial sequence
  Synthetic
ttgagaccatccaggattgg

(19) United States
(12) Patent Application Publication
(10) Pub. No.: US 2022/0301655 A1
(43) Pub. Date: Sep. 22, 2022

(54) SYSTEMS AND METHODS FOR GENERATING GRAPH REFERENCES

(71) Applicant: Seven Bridges Genomics Inc., Charlestown, MA (US)

(72) Inventors: Huseyin Serhat Tetikol, Somerville, MA (US); Deniz Turgut, Istanbul (TR)

(21) Appl. No.: 17/697,301

(22) Filed: Mar. 17, 2022

Related U.S. Application Data
(60) Provisional application No. 63/162,400, filed on Mar. 17, 2021.

(51) Int. Cl. G16B 20/20; G16B 20/40; G16B 30/20; G16B 45/00; G06T 11/20

(52) U.S. Cl. CPC G16B 20/20; G16B 20/40; G16B 30/20; G16B 45/00; G06T 11/206

(57) ABSTRACT

Techniques for generating a graph reference construct. The techniques include: obtaining a plurality of variants associated with a reference sequence construct; generating the graph reference construct using the plurality of variants and the reference sequence construct; and outputting the generated graph reference construct. Generating the graph reference construct includes: filtering the plurality of variants to obtain a filtered set of variants, the filtering including a first filtering stage and a second filtering stage, and generating the graph reference construct using the filtered set of variants. The first filtering stage includes identifying a first subset of variants at least in part by excluding one or more structural variants from the plurality of variants. The second filtering stage includes identifying the filtered set of variants at least in part by excluding one or more multiply-alignable variants from the first subset of variants.

### CROSS REFERENCE TO RELATED APPLICATIONS

This application claims the benefit of priority under 35 U.S.C. 119(e) to U.S. Provisional Patent Application Ser. No. 63/162,400, titled “SYSTEMS AND METHODS FOR GENERATING GRAPH REFERENCES”, and filed on Mar. 17, 2021, the entire contents of which are incorporated by reference herein.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Mar. 15, 2022, is named S196170030WO00-SEQ-DGR and is 5,033 bytes in size.

### BACKGROUND

Advances in sequencing technology, including the development of next generation sequencing methods, have made sequencing an important tool used in both research and in medicine. Some applications of sequencing technology include aligning the sequence reads obtained by sequencing techniques against a reference sequence construct, and identifying the differences, sometimes termed “variants,” between the sequence reads and the reference sequence construct. In turn, the identified differences may be used for diagnostic, prognostic, therapeutic, research, and/or other purposes.

There are different types of reference sequence constructs to which sequence reads may be aligned. For example, sequence reads may be aligned against a linear reference sequence construct such as, for example, the hg19 and hg38 human reference genomes. As another example, sequence reads may be aligned against a reference sequence construct that accounts for one or more known variants at one or more respective locations. One example of such a reference sequence construct is a graph-based reference sequence construct (sometimes referred to herein as a “graph reference construct”). A graph reference construct may include a graph (e.g., a directed acyclic graph) through which there may be multiple paths, each of which may represent one or multiple known variants.

### SUMMARY

Some embodiments provide for a method for generating a graph reference construct, the method comprising: using at least one computing device to perform: obtaining a plurality of variants associated with a reference sequence construct for at least a portion of a genome; and generating the graph reference construct using the plurality of variants and the reference sequence construct, the generating comprising: filtering the plurality of variants to obtain a filtered set of variants, the filtered set of variants being a subset of the plurality of variants, the filtering comprising a plurality of filtering stages including a first filtering stage and a second filtering stage different from and performed subsequent to the first filtering stage, the first filtering stage comprising identifying a first subset of variants from among the plurality of variants at least in part by excluding one or more structural variants from the plurality of variants, the one or more structural variants including a first structural variant; the second filtering stage comprising identifying the filtered set of variants from among the first subset of variants at least in part by excluding one or more multiply-alignable variants from the first subset of variants; generating the graph reference construct using the filtered set of variants and the reference sequence construct; and outputting the generated graph reference construct.

Some embodiments provide for a system, comprising: at least one computer hardware processor; and at least one non-transitory computer-readable storage medium storing processor-executable instructions that, when executed by the at least one computer hardware processor, cause the at least one computer hardware processor to perform: obtaining a plurality of variants associated with a reference sequence construct for at least a portion of a genome; generating the graph reference construct using the plurality of variants and the reference sequence construct, the generating comprising: filtering the plurality of variants to obtain a filtered set of variants, the filtered set of variants being a subset of the plurality of variants, the filtering comprising a plurality of filtering stages including a first filtering stage and a second filtering stage different from and performed subsequent to the first filtering stage, the first filtering stage comprising identifying a first subset of variants from among the plurality of variants at least in part by excluding one or more structural variants from the plurality of variants, the one or more structural variants including a first structural variant; the second filtering stage comprising identifying the filtered set of variants from among the first subset of variants at least in part by excluding one or more multiply-alignable variants from the first set of variants; and generating the graph reference construct using the filtered set of variants and the reference sequence construct; and outputting the generated graph reference construct.

Some embodiments provide for at least one non-transitory computer-readable storage medium storing processor-executable instructions that, when executed by at least one computer hardware processor, cause the at least one computer hardware processor to perform: obtaining a plurality of variants associated with a reference sequence construct for at least a portion of a genome; generating the graph reference construct using the plurality of variants and the reference sequence construct, the generating comprising: filtering the plurality of variants to obtain a filtered set of variants, the filtered set of variants being a subset of the plurality of variants, the filtering comprising a plurality of filtering stages including a first filtering stage and a second filtering stage different from and performed subsequent to the first filtering stage, the first filtering stage comprising identifying a first subset of variants from among the plurality of variants at least in part by excluding one or more structural variants from the plurality of variants, the one or more structural variants including a first structural variant; the second filtering stage comprising identifying the filtered set of variants from among the first subset of variants at least in part by excluding one or more multiply-alignable variants from the first set of variants; and generating the graph reference construct using the filtered set of variants and the reference sequence construct; and outputting the generated graph reference construct.

In some embodiments, identifying the first subset of variants from among the plurality of variants comprises: determining whether a first length of the first structural variant exceeds a first specified threshold; and upon determining that the first length exceeds the first specified threshold, excluding the first structural variant from the plurality of variants.

In some embodiments, the first structural variant is an insertion event, and determining whether the first length of the first structural variant exceeds the first specified threshold comprises determining whether the first length is at least 5,000 base pairs.

In some embodiments, the first structural variant is a deletion event, and determining whether the first length of the first structural variant exceeds the first specified threshold comprises determining whether the first length is at least 90,000 base pairs.

In some embodiments, identifying the first subset of variants from among the plurality of variants comprises: aligning the first structural variant to the reference sequence construct.

In some embodiments, identifying the first subset of variants from among the plurality of variants comprises: determining whether the reference sequence construct includes a subsequence, wherein the subsequence is identical to at least a portion of the first structural variant; and upon determining that the reference sequence construct includes the subsequence, excluding the first structural variant from the plurality of variants.

In some embodiments, identifying the first subset of variants from among the plurality of variants comprises: aligning the first structural variant to one or more variants of the plurality of variants, the one or more variants being different from the first structural variant.

In some embodiments, identifying the first subset of variants from among the plurality of variants comprises: determining whether a second structural variant includes a subsequence, wherein the subsequence is identical to at least a portion of the first structural variant; and upon determining that the second structural variant includes the subsequence, excluding one of the first structural variant or the second structural variant from the plurality of variants.

In some embodiments, identifying the first subset of variants from among the plurality of variants comprises: aligning the first structural variant to a decoy sequence associated with the reference sequence construct.

In some embodiments, identifying a first subset of variants from among the plurality of variants comprises: determining whether a decoy sequence associated with the reference sequence construct includes a subsequence, wherein the subsequence is identical to at least a portion of the first structural variant; and upon determining that the decoy sequence includes the subsequence, masking the decoy sequence.

In some embodiments, identifying the first subset of variants from among the plurality of variants further comprises, upon determining that the first length does not exceed the first specified threshold: determining whether the reference sequence construct includes a first subsequence, wherein the first subsequence is identical to at least a first portion of the first structural variant; and upon determining that the reference sequence construct includes the first subsequence, excluding the first structural variant from the plurality of variants.

In some embodiments, determining whether the reference sequence construct includes the first subsequence comprises determining whether the first subsequence has a length that is greater than a second specified threshold.

Some embodiments further comprise: upon determining that the reference sequence construct does not include the first subsequence, determining whether a second structural variant includes a second subsequence, wherein the second subsequence is identical to at least a second portion of the first structural variant; and upon determining that the second structural variant includes the second subsequence, excluding one of the first structural variant or the second structural variant from the plurality of variants.

In some embodiments, determining whether the second structural variant includes the second subsequence comprises determining whether the second subsequence has a length that is greater than the second specified threshold.

In some embodiments, the second specified threshold is at least 150 base pairs.

In some embodiments, excluding one of the first structural variant or the second structural variant from the plurality of variants comprises: identifying a shortest variant from among the first structural variant and the second structural variant; and excluding the shortest variant from the plurality of variants.

Some embodiments further comprise upon determining that the second structural variant does not include the second subsequence, determining whether a decoy sequence associated with the reference sequence construct includes a third subsequence, wherein the third subsequence is identical to at least a third portion of the first structural variant; and upon determining that the decoy sequence includes the third subsequence, masking the decoy sequence.

In some embodiments, identifying the filtered set of variants from among the first subset of variants comprises: generating an initial graph reference construct using at least some of the first subset of variants.

In some embodiments, identifying the filtered set of variants from among the first subset of variants further comprises: generating a plurality of graph reads using the initial graph reference construct, wherein each of at least some of the plurality of graph reads are associated with a respective path in the initial graph reference construct.

In some embodiments, the plurality of graph reads comprise a first subset of graph reads and a second subset of graph reads, and wherein generating the plurality of graph reads comprises: generating the first subset of graph reads by traversing the initial graph reference construct over a first interval; and generating the second subset of graph reads by traversing the initial graph reference construct over a second interval, wherein the first interval and the second interval at least partially overlap.

In some embodiments, generating the plurality of graph reads comprises traversing the initial graph reference construct using a sliding window with a skip.

Some embodiments further comprise aligning at least some of the plurality of graph reads to the initial graph reference construct, the aligning comprising, for each graph read of the at least some of the plurality of graph reads: determining a quality of alignment between the graph read and the graph reference construct; and determining whether the quality of alignment exceeds a threshold.

Some embodiments further comprise identifying a first group of the at least some of the plurality of graph reads, wherein each graph read included in the first group of the at least some of the plurality of graph reads includes a first combination of one or more variants of the first subset of variants.

In some embodiments, the first group of the at least some of the plurality of graph reads includes a first graph read and a second graph read; and further comprising: upon determining that neither a first quality of alignment determined for the first graph read nor a second quality of alignment determined for the second graph read exceed the specified threshold, excluding at least one multiply-alignable variant from the filtered set of variants.

In some embodiments, the at least one multiply-alignable variant is included in the first combination of the one or more variants.

In some embodiments, identifying the filtered set of variants from among the first subset of variants comprises: generating an initial graph reference construct using the first subset of variants; traversing the initial graph reference construct to generate a plurality of graph reads; aligning the plurality of graph reads to the initial graph reference construct to determine qualities of alignment for each of at least some of the plurality of graph reads; and excluding at least some of the one or more of the first set variants from the second set of variants based on the qualities of alignment.

In some embodiments, one or more of the plurality of graph reads are associated with a same combination of one or more of the first subset of variants. Some embodiments further comprise: determining whether each of the qualities of alignment determined for the one or more of the plurality of graph reads is below a specified threshold; and upon determining that each of the qualities of alignment is below the specified threshold, excluding at least one variant from the filtered set of variants.

In some embodiments, obtaining the plurality of variants comprises: obtaining a plurality of alternative sequences associated with the reference sequence construct; processing at least some of the plurality of alternative sequences, the processing comprising, for a first alternative sequence of the plurality of alternative sequences: aligning the first alternative sequence to the reference sequence construct to obtain an aligned position; identifying one or more differences between the first alternative sequence and the reference sequence construct at the aligned position; and including at least some of the one or more differences as first variants in the plurality of variants.

In some embodiments, processing the at least some of the plurality of alternative sequences, constructing an updated reference sequence construct that does not include the plurality of alternative sequences.

In some embodiments, the first alternative sequence includes an inverted sequence patch; and wherein aligning the first alternative sequence to the reference sequence construct to obtain the aligned position comprises obtaining an alternative aligned position for the inverted sequence patch.

Some embodiments further comprise left normalizing the first variants with respect to the reference sequence construct before including the first variants in the plurality of variants.

In some embodiments, the at least some of the one or more differences include consecutive first and second differences, wherein the first difference is associated with a first subsequence of the first alternative sequence, and wherein the second difference is associated with a second subsequence of the reference sequence construct. Some embodiments further comprise processing the first and second differences before including them as first variants in the plurality of variants, the processing comprising: determining whether the first subsequence includes one or more regions that are included in the second subsequence; and upon determining that the first subsequence includes the one or more regions that are included in the second subsequence, removing the one or more regions from both the first and second subsequences.

In some embodiments, the first and second differences respectively comprise insertion and deletion events.

In some embodiments, obtaining the plurality of variants further comprises: obtaining second variants associated with the reference sequence construct; and including the second variants in the plurality of variants.

Some embodiments further comprise annotating the second variants with information indicative of sources of the second variants.

In some embodiments, at least some of the first variants are associated respectively with first allele frequencies and at least some of the second variants are associated respectively with second allele frequencies. Some embodiments further comprise, for a shared variant included in both the at least some of the first variants and the at least some of the second variants, averaging the first and second allele frequencies associated with the shared variant to obtain an averaged allele frequency.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a diagram of an illustrative technique for generating a graph reference construct, according to some embodiments of the technology described herein (SEQ ID NOs: 1-2).

FIG. 2A is a flowchart of an illustrative process 200 for generating a graph reference construct, according to some embodiments of the technology described herein.

FIG. 2B is a flowchart illustrating an example process 220 for processing variants associated with a reference sequence construct, according to some embodiments of the technology described herein.

FIG. 2C is a flowchart illustrating an example process 240 for processing a structural variant, according to some embodiments of the technology described herein.

FIG. 2D is a flowchart illustrating an example process 260 for identifying the filtered set of variants from among the first subset of variants, according to some embodiments of the technology described herein

FIG. 3A is an illustrative example of processing alternate sequences associated with a reference construct, according to some embodiments of the technology described herein (SEQ ID NOs: 3-4).

FIG. 3B is a diagram of an illustrative example of performing a first stage of a multi-stage variant filtering technique, with the first stage being used to identify a set of structural variants to be excluded from an initial set of variants, according to some embodiments of the technology described herein (SEQ ID NOs: 5-12). FIG. 3C is a diagram of an illustrative example of performing a second stage of a multi-stage variant filtering technique, with the second stage being used to identify a set of multiply-alignable variants to be excluded from the initial set of variants, according to some embodiments of the technology described herein (SEQ ID NOs: 13-23).

FIG. 4A is a diagram depicting an exemplary process 400 for generating a graph reference construct, according to some embodiments of the technology described herein.

FIG. 4B is a diagram depicting an exemplary process 402 for processing alternate sequences associated with a reference sequence construct, according to some embodiments of the technology described herein.

FIG. 4C is a diagram depicting an exemplary process 422 for identifying a set of structural variants, according to some embodiments of the technology described herein.

FIG. 4D is a diagram depicting an exemplary process 424 for identifying a set of multiply-alignable variants, according to some embodiments of the technology described herein.

FIG. 5 shows graphs depicting alignment metrics from an experiment to measure the performance of graph reference constructs, according to some embodiments of the technology described herein.

FIG. 6 shows graphs depicting variant calling metrics from an experiment to measure the performance of graph reference constructs, according to some embodiments of the technology described herein.

FIG. 7 shows graphs depicting cumulative variant counts with respect to allele frequency from an experiment to measure the performance of graph reference constructs, according to some embodiments of the technology described herein.

FIG. 8 shows graphs depicting the variant counts from an experiment to measure the performance of graph reference constructs, according to some embodiments of the technology described herein.

FIG. 9 is a block diagram of an illustrative computer system that may be used in implementing some embodiments of the technology described herein.

### DETAILED DESCRIPTION

Aligning sequence reads against a graph reference construct, which accounts for known genetic variations among people, aids accurate placement of sequence reads and facilitates identification of variants based on results of alignment. However, the inventors have recognized and appreciated that conventional techniques for aligning sequence reads against graph reference constructs may be improved upon because conventional techniques may produce inaccurate results and are computationally expensive.

Aligning sequence reads against a graph reference construct may lead to inaccurate results when the graph reference construct includes all curated variants (e.g., variants selected to represent genetic variation,) without considering how those variants may affect alignment. First, the curated variants may include structural variants. A structural variant may include an insertion of at least a threshold length (e.g., at least 40 bp, at least 50 bp, at least 60 bp, at least 80 bp, at least 100 bp, at least 150 bp, at least 500 bp, at least 1K bp, at least 5K bp, at least 20K bp, at least 50K bp, at least 100K bp, at least 500K bp, etc.), a deletion of at least a threshold length (e.g., at least 40 bp, at least 50 bp, at least 60 bp, at least 80 bp, at least 100 bp, at least 150 bp, etc.), an inversion of at least a threshold length (e.g., at least 40 bp, at least 50 bp, at least 60 bp, at least 80 bp, at least 100 bp, at least 150 bp, at least 500 bp, at least 1K bp, at least 5K bp, at least 20K bp, at least 50K bp, at least 100K bp, at least 500K bp, etc.), a duplication of at least a threshold length (e.g., at least 40 bp, at least 50 bp, at least 60 bp, at least 80 bp, at least 100 bp, at least 150 bp, at least 500 bp, at least 1K bp, at least 5K bp, at least 20K bp, at least 50K bp, at least 100K bp, at least 500K bp, etc.), and/or any other suitable structural variant. Structural variants may introduce ambiguity to the graph reference construct due to the nature of short-read sequencing data. In other words, if a structural variant includes a subsequence that is (a) identical to other portions of the graph reference and (b) longer than a sequence read, the sequence read may be incorrectly aligned to more than one position in the graph reference construct. Second, as more variants are incorporated in the graph reference construct, the number of possible paths in the graph grows exponentially, increasing the likelihood that there will be identical paths in different regions of the graph. As a result, a sequence read may be aligned to multiple regions in the graph reference construct, becoming uninformative for variant calling. Such variants may be referred to herein as “multiply-alignable variants.”

Additionally, the curated variants may be obtained from multiple different sources, such as multiple variant databases or VCF files. As a result of the discordance between the variant representation of different bioinformatic pipelines, the same variants may be expressed differently when obtained from different sources. Addition of such variants may introduce different but ultimately equivalent paths in the graph reference, resulting in alignment inaccuracies.

Further, aligning sequence reads to such a graph reference construct may be computationally expensive, since the curated variants may include many variants from many individuals. Since a known variant in a graph reference may be represented by a respective path through the graph underlying the graph reference, increasing the number of known variants represented by a graph reference increases the number of paths through the graph that have to be evaluated during alignment of sequence reads to the graph reference, which in turn increases the computational complexity of performing the alignment. Moreover, added complexity in the structure of the graph reference may result in noise during alignment, reducing accuracy.

Accordingly, the inventors have developed techniques for generating a graph reference construct that excludes variants (e.g., structural variants and/or multiply-alignable variants) that cause alignment ambiguity, leading not only to more accurate alignment results, but also reducing the overall computational complexity of such alignments. In some embodiments, a set of variants may be filtered in multiple stages to identify the variants that are included in the graph reference construct. For example, different stages of filtering may include filtering out different types of variants (e.g., structural variants may be filtered out in one stage and multiply-alignable variants may be filtered out in another stage, for example, in a subsequent stage to the stage in which the structural variants are filtered.) In some embodiments, the identified variants may be used to construct the graph reference construct, for example, by adding nodes and edges representing the filtered set of variants to a linear reference construct.

Some embodiments provide for computer-implemented techniques for generating a graph reference construct (e.g., a directed acyclic graph (DAG)). In some embodiments, the techniques include: (A) obtaining a plurality of variants associated with a reference sequence construct for at least a portion of the genome (e.g., at least a substantial portion, at least a chromosome, at least 10,000 nucleotides, etc.); (B) generating the graph reference construct using the plurality of variants and the reference sequence construct (e.g., the hg19 or hg38 genome references,) and (C) outputting the generated graph reference construct (e.g., saving the graph reference construct to memory so that it can be used subsequently for various applications including, for example, for aligning sequence reads against the graph reference construct, etc.). In some embodiments, techniques for generating the graph reference construct include: (A) filtering the plurality of variants to obtain a filtered set of variants, the filtered set of variants being a subset of the plurality of variants, the filtering including a plurality of filtering stages including a first filtering stage (e.g., for excluding variants of a first type) and a second filtering stage (e.g., for excluding variants of a second type) different from and performed subsequent to the first filtering stage; and (B) generating the graph reference construct using the filtered set of variants (the filtered set of variants by applying the first and second filtering stages) and the reference sequence construct.

In some embodiments, the first filtering stage includes identifying a first subset of variants from among the plurality of variants at least in part by excluding one or more structural variants (e.g., insertion events, deletion events, or inversion events) from the plurality of variants. In some embodiments, the second filtering stage includes identifying the filtered set of variants from among the first subset of variants (e.g., variants identified in the first filtering stage) at least in part by excluding one or more multiply-alignable variants (e.g., variants that result in multi-mapped sequence reads) from the first subset of variants.

It should be appreciated that the techniques described herein may be implemented in any of numerous ways, as the techniques are not limited to any particular manner of implementation. Examples of details of implementation are provided herein solely for illustrative purposes. Furthermore, the techniques disclosed herein may be used individually or in any suitable combination, as aspects of the technology described herein are not limited to the use of any particular technique or combination of techniques.

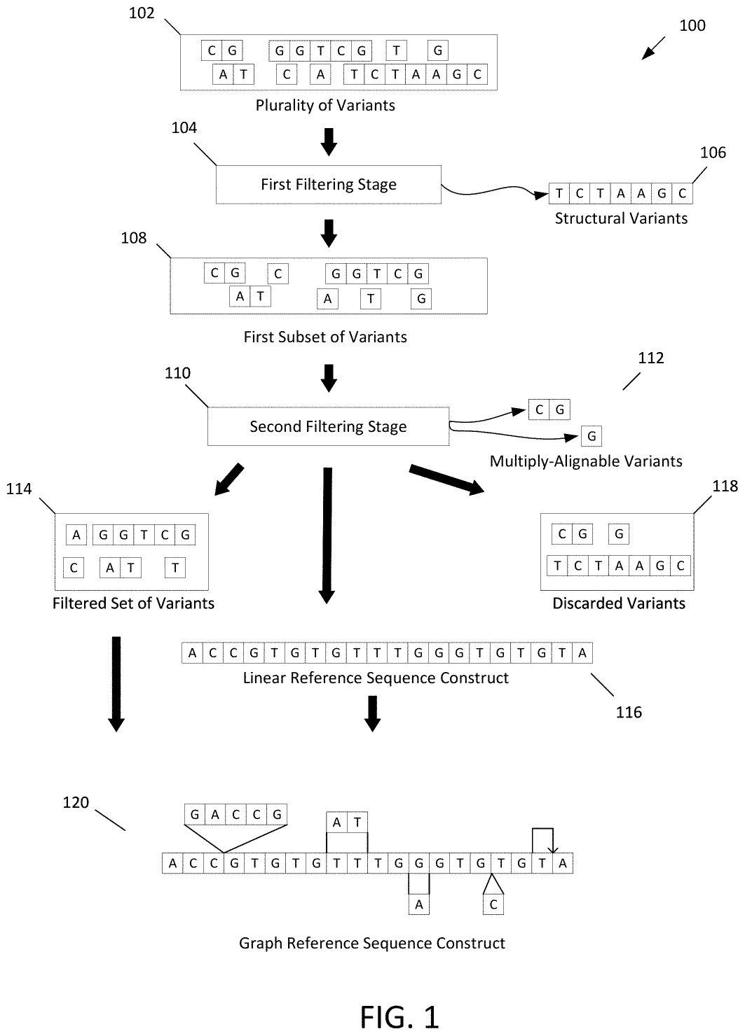

Some illustrative aspects of the technology described herein are described below with reference to FIGS. 1-9.

FIG. 1 is a diagram of an illustrative technique 100 for generating a graph reference construct, according to some embodiments of the technology described herein. In some embodiments, the illustrative technique 100 involves obtaining a plurality of variants 102. Using a first filtering stage 104, one or more structural variants 106 may be identified and excluded from the plurality of variants 102, resulting in the first subset of variants 108. Using a second filtering stage 110, one or more multiply-alignable variants 112 may be identified and excluded from the first subset of variants 108 to obtain the filtered set of variants 114. In some embodiments, the output of the second filtering stage 110 includes the filtered set of variants 114, the discarded set of variants 118 (e.g., excluded during the first and second filtering stages), and/or a linear reference sequence construct 116. In some embodiments, variants included in the filtered set of variants 114 and the linear reference sequence construct 116 are used to construct a graph reference sequence construct.

In some embodiments, obtaining the plurality of variants 102 includes obtaining variants from one or more sources. In some embodiments, this includes obtaining variants from one or more publicly available variant databases and/or variant call format (VCF) files. For example, the plurality of variants may be obtained from the GRCh38 human reference alternate contigs, 1000 Genome Project common variants, Simons Genome Diversity Project common variants, the Human Genome Structural Variant Consortium (HGSVC) and/or any other suitable variant databases and/or VCF files.

In some embodiments, the plurality of variants 102 are associated with a reference sequence construct. For example, the reference sequence construct may include the GRCh38 genome assembly. In some embodiments, the reference sequence construct is constructed using primary chromosomes, decoys, and alternate sequences that represent divergences from the primary assembly. A decoy may include common additional sequences that are not in the reference. In some embodiments, if decoy sequences are not included in the reference sequence construct, then sequence reads may incorrectly map to regions of the primary chromosome. For example, the HS38D1 and EBV decoys may be included in the reference sequence construct.

In some embodiments, the first filtering stage 104 involves identifying and excluding one or more structural variants 106 from the plurality of variants to identify a first subset of variants. In some embodiments, the first filtering stage 104 includes evaluating a variant in multiple stages to determine whether including the variant in the graph construct could (a) be too computationally expensive for sequence alignment, and/or (b) result in incorrect sequence alignment.

In some embodiments, including structural variants in the graph reference construct increases the computational complexity of aligning to such a graph reference construct. In some embodiments, the first filtering stage 104 includes excluding structural variants that are too large. For example, insertions that are greater than a threshold size (e.g., greater than 1K, 2K, 3K, 5K, 10K, 15K, 20K, 25K, any number of base pairs in the range 1-25K) may be excluded from the plurality of variants. As another example, deletions that are greater than a threshold size (e.g., greater than 50K, 70K, 90K, 100K, 110K, 150K, 200K, 250K, 300K, any number of base pairs in the range 50K-300K) may be excluded at the first filtering stage. In some embodiments, the threshold sizes of different structural variants vary based on the characteristics of the aligner. In some embodiments, excluding these large structural variants from the plurality of variants makes alignment computationally feasible and substantially more computationally efficient, whereas without removing such structural variants, the cost of aligning sequence reads to the resulting graph is computationally expensive or, in some instances, infeasible.

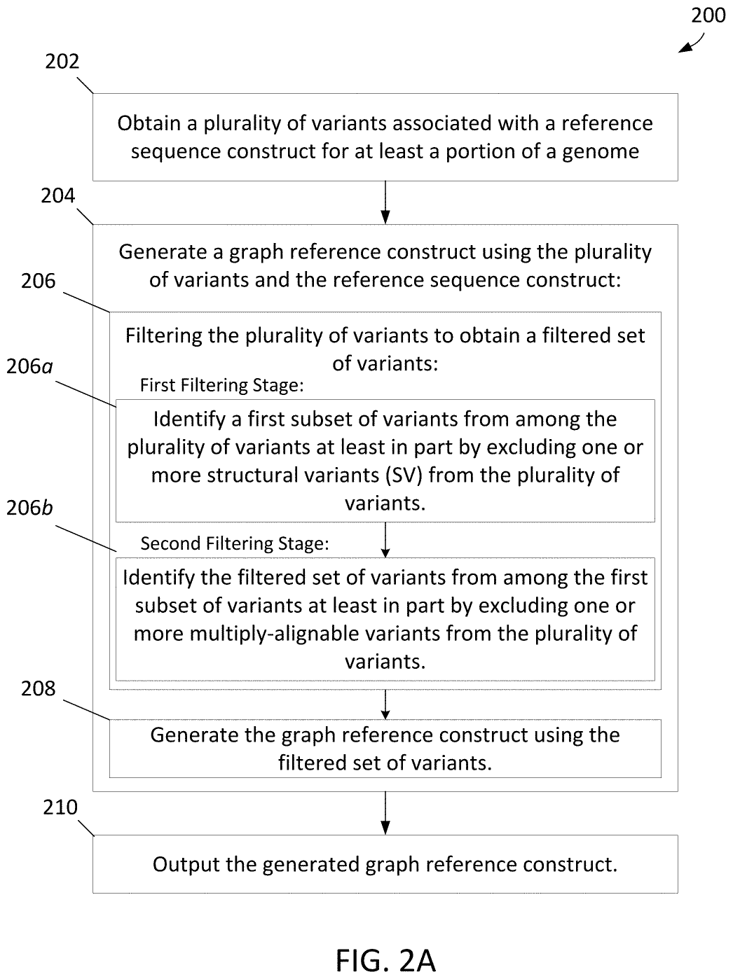

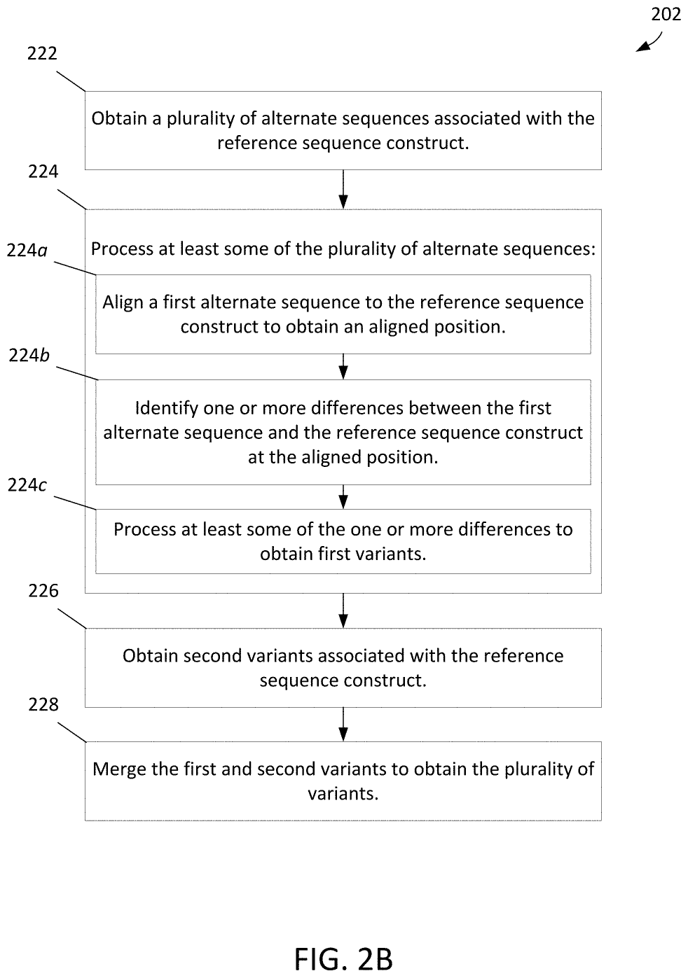

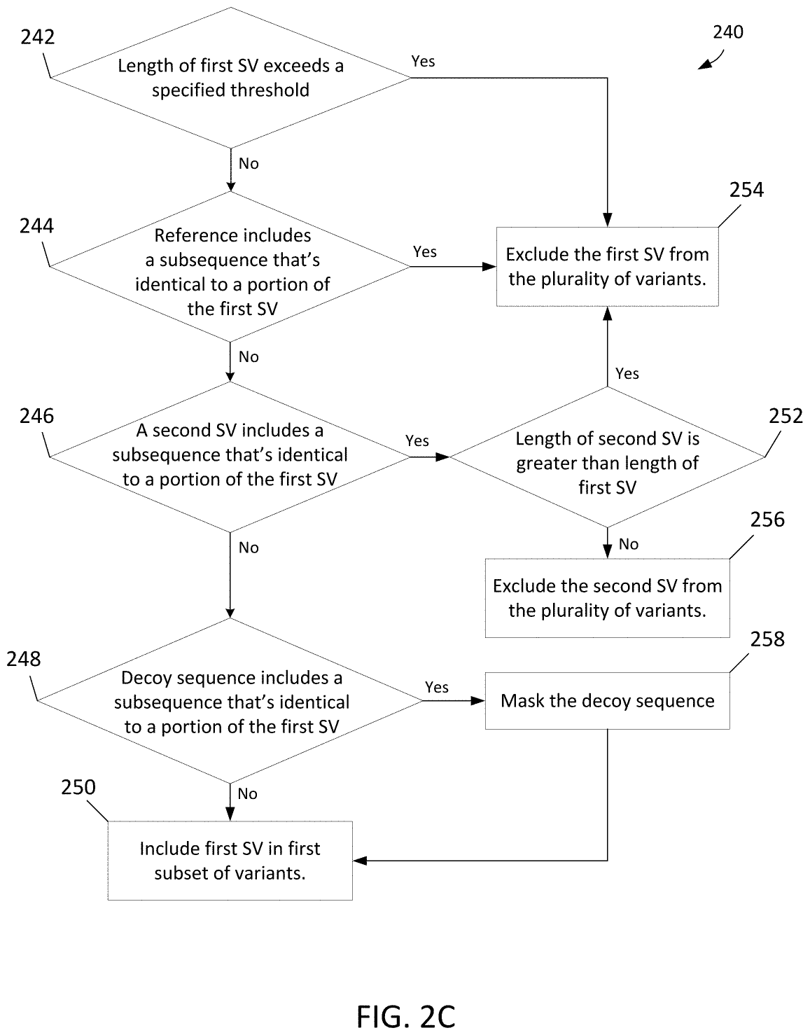

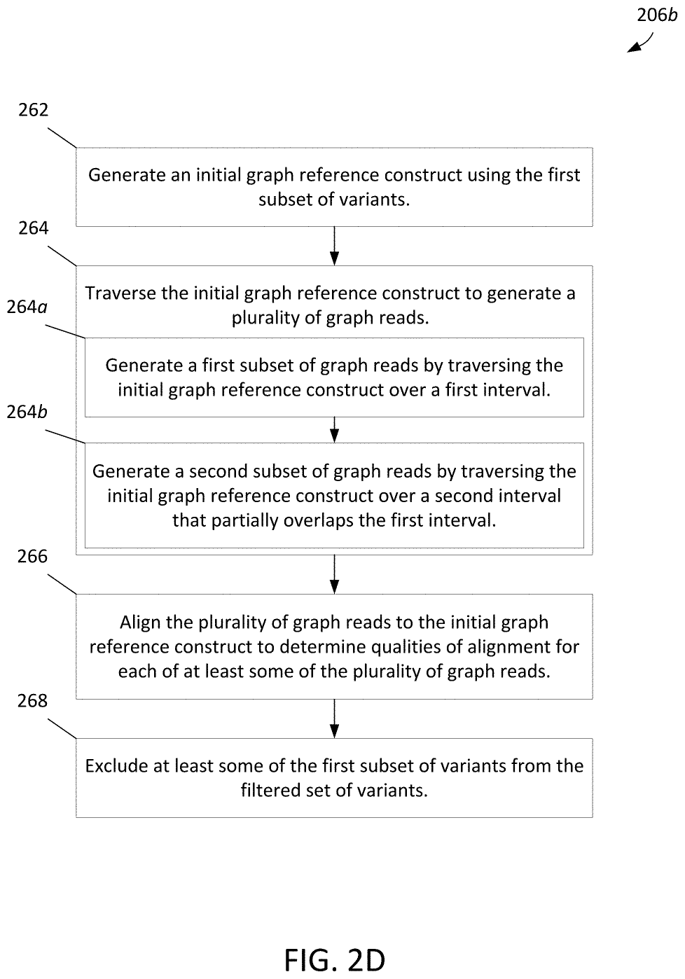

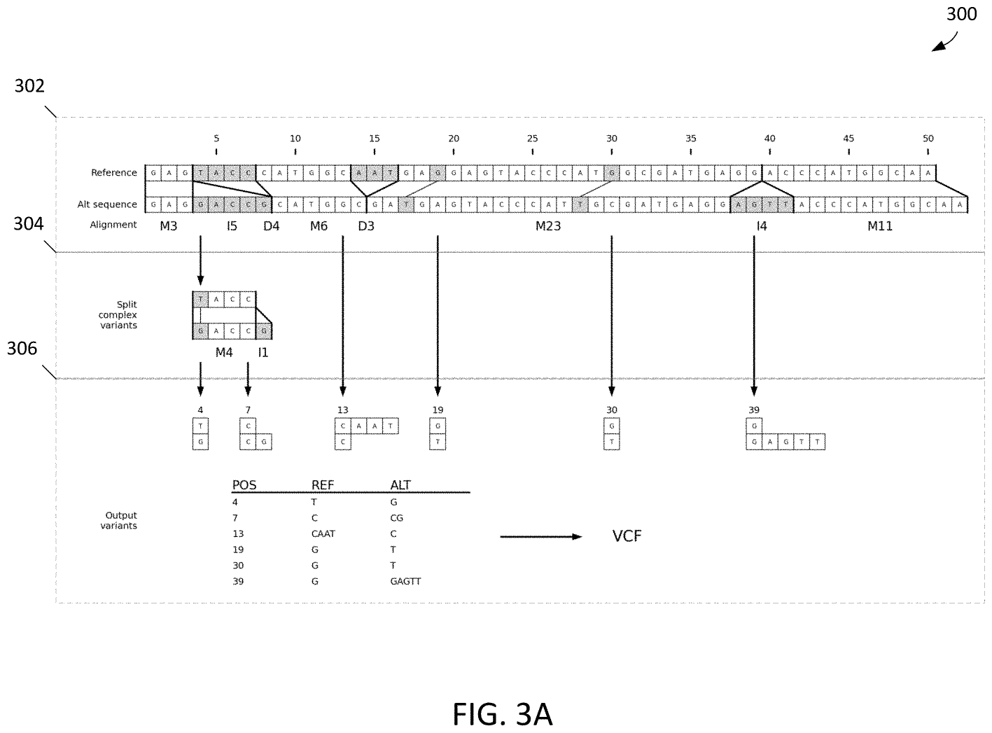

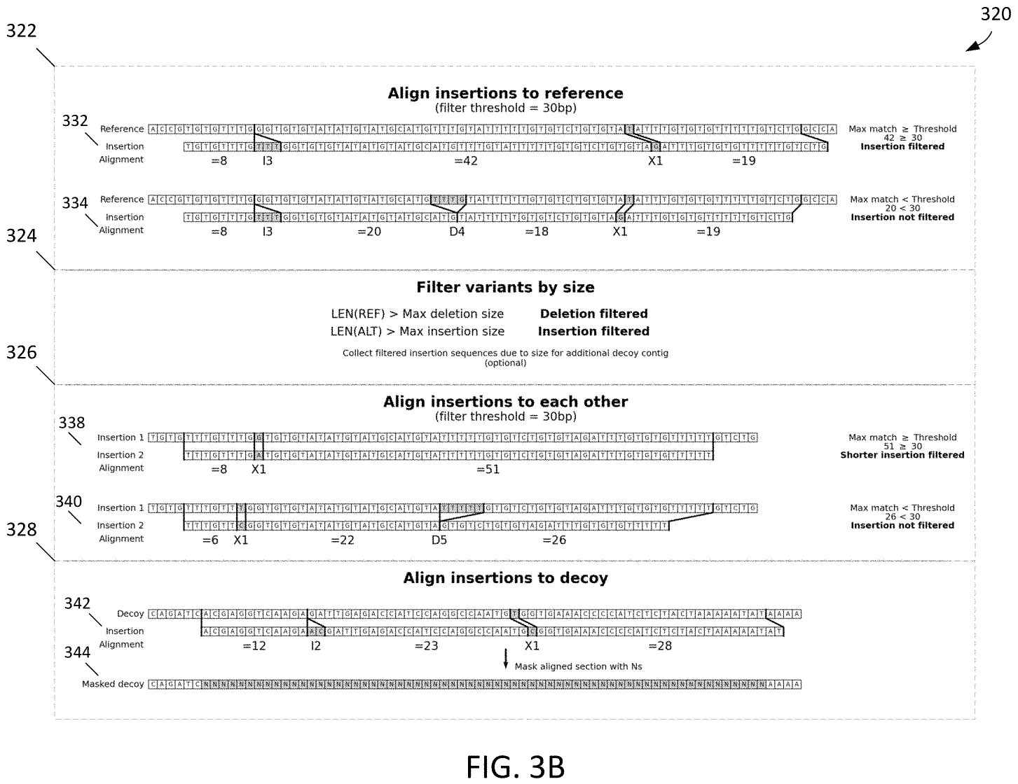

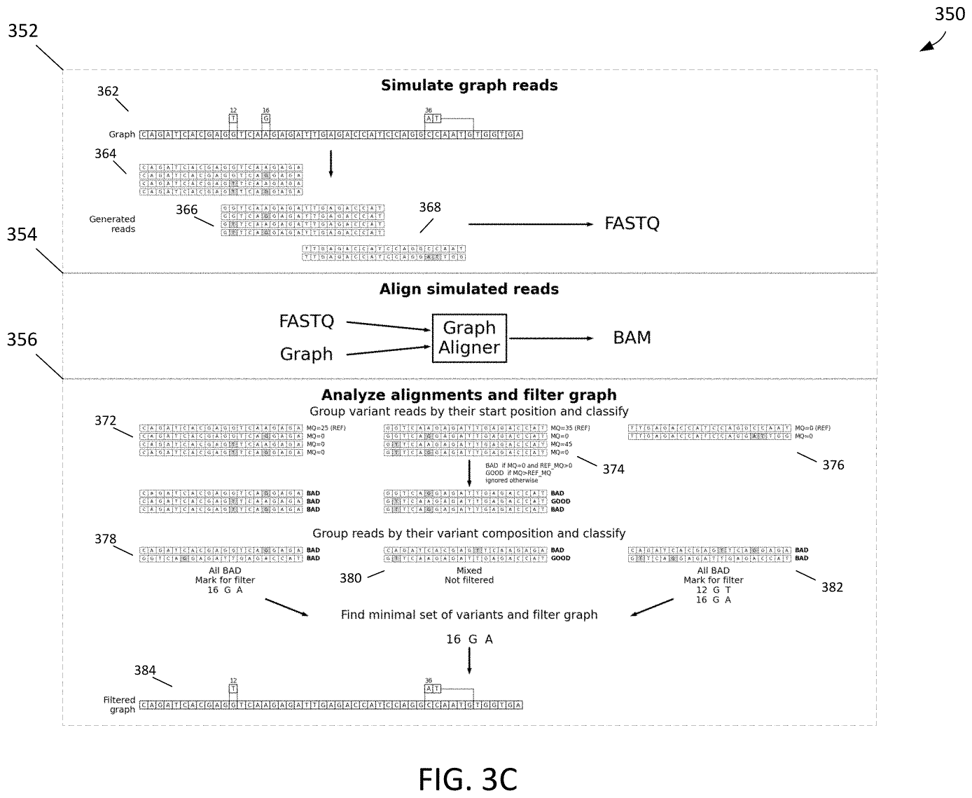

In some embodiments, a structural variant that includes a subsequence that is (a) identical to another subsequence included in the graph reference construct (e.g., another variant, the linear reference construct, or a decoy sequence) results in inaccurate or ambiguous alignment. For example, if a sequence read is shorter in length than such a repeated subsequence, the sequence read may be aligned to each of those subsequences or incorrectly aligned to one of those subsequences. Therefore, in some embodiments, the first filtering stage 104 includes determining whether a structural variant includes a subsequence that is identical to a subsequence included in the reference sequence construct, other variants included in the plurality of variants, and/or decoy sequences associated with the reference sequence construct. If it is determined that the structural variant includes a subsequence identical to a subsequence included in the reference sequence construct, and that the subsequence has a length that exceeds a specified threshold (e.g., the length of a sequence read), then the structural variant may be excluded from the plurality of variants. If it is determined that the structural variant includes a subsequence that is included in another variant (e.g., another structural variant), and that the subsequence has a length that is greater than the specified threshold, then the shorter of the two variants may be excluded from the plurality of variants. If it is determined that the structural variant includes a subsequence that is included in a decoy sequence, then that subsequence is masked in the decoy sequence. In some embodiments, each of these determinations (e.g., with respect to the reference sequence construct, other variants, and decoy sequence) may be made, some of these determinations may be made, or only one of these determinations may be made. Aspects of identifying a first subset of variants using a first filtering stage are described herein including at least with respect to FIGS. 2C and 3B.

In some embodiments, the second filtering stage 110 involves identifying and excluding one or more multiply-alignable variants 112 from the first subset of variants 108 to obtain a filtered set of variants 114. A “multiply-alignable” variant may be a variant that, when incorporated into a graph reference construct, results in two or more identical paths in different non-contiguous regions in the graph reference construct. For example, incorporating a multiply-alignable variant into a graph reference construct may result in a first path at a first region of the graph reference construct that is identical to a second path at a second region of the graph reference construct, where the first path includes at least a portion (e.g., at least some or all) of the multiply-alignable variant. Because a multiply-alignable variant may result in two or more identical paths in the graph reference construct, sequence reads that aligns to one path in the graph reference construct may also align to at least one other path the graph reference construct. Hence the name “multiply-alignable”—such variants may cause sequence reads to align to multiple regions in the graph reference construct.

In some embodiments, the second filtering stage 110 involves evaluating whether including one or more variants in a graph reference construct will result in two or more identical paths in different (e.g., non-contiguous) regions of the graph reference construct (e.g., evaluating whether the one or more variants are multiply-alignable variants). In some embodiments, aligning a sequence read against a graph reference construct that includes identical paths in different regions (e.g., a graph reference construct that includes multiply-alignable variants) may result in multi-mapped reads, which will then be uninformative for variant calling.

In some embodiments, the second filtering stage 110 involves generating multiple graph reads using an initial graph reference construct that includes the first subset of variants 108. A graph read may represent a sequence at a particular region of the initial graph reference construct. In turn, one or more of the graph reads may be each aligned to the initial graph reference to determine a respective mapping quality. The resultant mapping qualities may be indicative of the probability that the alignment is correct. The mapping qualities can then be used to identify multiply-alignable variants. For example, when aligning a graph read results in a low mapping quality (e.g., a mapping quality of 0), this may indicate that the graph read aligns to multiple regions in the initial graph reference construct. In some embodiments, multiple graph reads may represent a same variant or a same combination of variants. In this case, if aligning each of those graph reads result in a low mapping quality, it is likely that the shared variant or combination of variants introduces one or more identical path(s) in the initial graph reference construct. As a result, the second filtering stage 110 may include excluding one or more of the shared variants (e.g., multiply-alignable variants) 112 from the first subset of variants 108 to obtain the filtered set of variants 114. Aspects of identifying a filtered set of variants using a second filtering stage are described herein including at least with respect to FIGS. 2D and 3C.

In some embodiments, the linear reference sequence construct 116 includes a linear human genome reference. For example, the linear reference sequence construct 116 may include the hg19 or hg38 human genome reference. In some embodiments, the linear reference sequence construct 116 may have undergone one or more stages of processing. For example, as described herein, including with respect to FIG. 2B, one or more alternate sequences may be removed from the linear reference sequence construct. As another example, one or more of the discarded variants 118 (e.g., one or more of the multiply-alignable variants 112) may be included as decoy sequences associated with the linear reference sequence construct 116. In some embodiments, the linear reference sequence construct 116 may be output as one or more files (e.g., one or more VCF files.)

In some embodiments, generating a graph reference sequence construct 116 may include transforming the linear reference construct 116 into a graph reference by adding nodes and edges representing genetic variation. For example, a linear reference construct may be transformed into a graph reference by adding nodes and edges representing the filtered set of variants 114. Techniques for adding nodes and edges to a linear reference construct based on a set of variants are described in U.S. Pat. Pub. No.: 2015-0057946, titled “METHODS AND SYSTEMS FOR ALIGNING SEQUENCES,” published on Feb. 26, 2015, which is incorporated by reference herein in its entirety.

FIG. 2A is a flowchart of an illustrative process 200 for generating a graph reference construct, according to some embodiments of the technology described herein.

In some embodiments, process 200 begins at act 202 where obtaining a plurality of variants associated with a reference sequence construct for at least a portion of the genome is performed. In some embodiments, obtaining the plurality of variants includes accessing one or more variant databases and/or VCF files. For example, this may include accessing the GRCh38 human reference alternate contigs, 1000 Genome Project common variants, Simons Genome Diversity Project common variants, the Human Genome Structural Variant Consortium (HGSVC) and/or any other suitable variants from any suitable variant database, data store, file, and/or VCF file. In some embodiments, variants obtained from different databases and/or files may contain variants from a variety of population studies. In some embodiments, different variant files may include the same variant or set of variants. Techniques for obtaining the plurality of variants are described herein including at least with respect to FIG. 2B.

In some embodiments, variants may be associated with a reference sequence construct, such as the GRCh38 genome assembly. In some embodiments, the reference sequence construct represents at least a portion of a genome. For example, the reference sequence construct may represent at least a substantial proportion of a genome (e.g., 80% of a genome), at least a chromosome, at least 10,000 nucleotides, or approximately the entire genome of a particular organism. In some embodiments, a variant that is associated with the linear reference construct is defined in the context of the reference sequence construct, much like a coordinate system. For example, a variant may be represented by an identifier (e.g., a unique alphanumeric, alphabetic, or numeric character) that identifies a position of the variant with respect to the reference sequence construct. Techniques for obtaining the plurality of variants are further described herein including at least with respect to FIG. 2B.

After obtaining the plurality of variants, process 200 proceeds to act 204 where generating a graph reference construct using the plurality of variants and the reference sequence construct is performed. As described herein, in some embodiments, aligning a sequence read to a graph reference construct that includes all the variants obtained at act 202 may result in inaccurate or ambiguous alignment and may be computationally expensive. Therefore, as shown in FIG. 2A, act 204 may include filtering the plurality of variants to obtain a filtered set of variants. In some embodiments, filtering the variants includes a first filtering stage 206a and a second filtering stage 206b.

In some embodiments, the first filtering stage 206a includes identifying a first subset of variants from among the plurality of variants by excluding one or more structural variants from the plurality of variants. For example, the structural variants may include one or more insertions, deletions, inversions, duplications, or translocations of at least 50 bp in length. In some embodiments, identifying the first subset of variants includes identifying one or more structural variants for exclusion from the plurality of variants. An example for processing one such structural variant is described herein including at least with respect to process 240 shown in FIG. 2C. In some embodiments, process 240 may be repeated to process multiple.

In some embodiments, the second filtering stage 206b includes identifying a second subset of variants from among the plurality of variants by excluding one or more multiply-alignable variants from the plurality of variants. For example, in the case that the first subset of variants is included in the graph reference construct, the second filtering stage may include determining whether a path in one region of the graph reference construct is identical to one or more paths in one or more other regions of the graph reference. In some embodiments, if identical paths are identified, variants that give rise to such paths (e.g., multiply-alignable variants) may be excluded from the graph to obtain a unique set of paths in the graph. An example implementation of act 206b is described herein including with respect to FIG. 2D.

In some embodiments, after obtaining the filtered set of variants at act 206, process 200 proceeds to act 208 where generating the graph reference construct is performed using the filtered set of variants. In some embodiments, generating the graph reference construct may include adding one or more nodes or edges representing the filtered set of variants to the reference sequence construct.

At act 210, the generated graph reference construct may be output. In some embodiments, outputting the graph reference construct may include storing the graph reference construct such that it may be used subsequently for one or more applications (e.g., for aligning sequence reads to the graph reference construct in any subsequent bioinformatics pipeline). For example, the generated graph reference construct may be stored locally on the computing device used to perform process 200 (e.g., on a non-transitory storage medium coupled to or part of the computing device). In some embodiments, the graph reference construct may be stored in one or more external storage mediums (e.g., such as a remote database or cloud storage environment). The stored graph reference construct may be used subsequently, for example, to align sequence reads against the graph reference construct. FIG. 2B is a flowchart illustrating a process 220 for processing variants associated with a reference sequence construct, according to some embodiments of the technology described herein. Process 220 is an example for how act 202 of process 200 may be implemented.

As shown, process 220 begins at act 222 for obtaining a plurality of alternate sequences associated with the reference sequence construct for at least a portion of a genome. Alternate sequences, or alternate contigs, represent genetic divergence from the reference sequence construct (e.g., the primary assembly.) As such, there are nucleotide sequence differences between the alternate sequence and the corresponding portion of the reference sequence construct. In some embodiments, the alternate sequence may be highly diverged (e.g., at least 80% novel) from the corresponding portion of the reference sequence construct. In some embodiments, the alternate sequence may be very similar (e.g., differing by a few nucleotides) to the corresponding portion of the reference sequence construct.

In some embodiments, obtaining the alternate sequences at act 222 includes obtaining one or more files that describe the alignment of alternate sequences with respect to the reference sequence construct. For example, when using the GRCh38 assembly as the reference sequence construct, this may include obtaining one or more files from general feature format (GFF) files that describe the alignment of alternate sequences with respect to the primary chromosome. In some embodiments, the files describe the alignment of the alternate sequences in any suitable format. For example, the files may describe the alignment of the alternate sequences in concise idiosyncratic gapped alignment report (CIGAR) format. However, it should be appreciated that the alternate sequences may be obtained from any suitable source (e.g., database, file, etc.) in any suitable format, as aspects of the technology described herein are not limited in this respect.

In some embodiments, the reference sequence construct includes the alternate sequences as part of the primary assembly. Since aspects of the technology described herein include adding at least some processed alternate sequences to the reference sequence construct to obtain a graph reference construct, the alternate sequences may be removed from the primary assembly.

As described above, some of the alternate sequences may be very similar to the reference sequence construct. In particular, some of the alternate sequences may include large subsequences that are identical to subsequences included in the reference sequence construct. As a result, incorporating the alternate sequences into a graph reference construct may cause short sequence reads to incorrectly align to the multiple identical regions. Therefore, process 220 includes techniques for addressing such concerns. Specifically, act 224 includes processing at least some of the alternate sequences obtained at act 222. In some embodiments, processing the alternate sequences includes sub-acts 224a, 224b, and 224c.

As shown in FIG. 2B, sub-act 224a includes aligning a first alternate sequence to the reference sequence construct to obtain an aligned position for the first alternate sequence. In some embodiments, the alignment may be performed using any suitable alignment technique, as aspects of the technology described herein are not limited in this respect. For example, in some embodiments, alignment may be performed using any of the techniques described in U.S. Pat. Pub. No.: 2015-0057946, titled “METHODS AND SYSTEMS FOR ALIGNING SEQUENCES,” published on Feb. 26, 2015, which is incorporated by reference herein in its entirety. In some embodiments, an aligned position may have been previously obtained for the alternate sequences, making sub-act 224a optional. For example, as described above, the one or more files obtained at act 222 may describe an alignment of the alternate sequences with respect to the reference sequence construct.

After the first alternate sequence is aligned at sub-act 224a, process 220 proceeds to sub-act 224b for identifying one or more differences between the first alternate sequence and the reference sequence construct at the aligned position. In some embodiments, the one or more differences include one or more nucleotide sequence differences. In some embodiments, the one or more differences may be sequence variants, such as substitutions, insertions, deletions, translocations, inversions, or any other suitable type of sequence mutation or variant. For example, the reference sequence construct may include the subsequence “AGGTCA” while an aligned alternate sequence includes the subsequence “AAGTCA.” The “G” at the second position of the reference subsequence is substituted for the “A” at the second position of the alternate subsequence. In some embodiments, the one or more differences at sub-act 224b may be identified using any suitable techniques. For example, the techniques may include processing one or more files describing the alignment in CIGAR (or any other) format and extracting the differences.

In some embodiments, an alternate sequence might contain an inverted sequence patch. For example, regions on either side of the alternate sequence patch may align to the reference sequence construct in a forward direction, while the inverted sequence patch aligns to the reference sequence construct in the reverse direction. In some embodiments, the techniques include obtaining an alternative alignment for an inverted sequence patch, then extracting the one or more differences from the alternative alignment.

In some embodiments, the portions of the first alternate sequence that are not identified at act 224b are excluded from further processing. For example, portions of the first alternate sequence that are identical to the reference sequence construct may be excluded from further processing. By contrast, in some embodiments, the one or more differences identified at act 224b are included during further processing. Not only does this reduce computational complexity (e.g., due to the large sizes of some alternate sequences prior to excluding identical portions), but it also improves accuracy of sequence read alignment. For example, if identical subsequences are not excluded from the graph reference construct, a sequence read may inaccurately align to both subsequences.

After identifying one or more differences between the reference sequence construct and the first alternate sequence, the example implementation proceeds to sub-act 224c, where at least some the one or more differences are processed to obtain variants. In some embodiments, the differences may include consecutive differences, such as consecutive insertion and deletion events. Sometimes, the consecutive differences may include identical subsequences, which may be identified by aligning differences against one another. Consider an example insertion event that includes the nucleotides “AGGTCGA” and an example, consecutive deletion event that includes the nucleotides “CCGTCGG.” After aligning the events against one another, using Needleman-Wunsch algorithm, for example, the subsequence “GTCG” is identified as a matching subsequence (e.g., included in both the insertion and the deletion event). In some embodiments, sub-act 224c includes excluding the matching subsequence and splitting both differences into smaller variations. In this example, excluding the matching subsequence would result in insertions “AG” and “A” and would result in deletions of “CC” and “G.” An example of processing differences to exclude matching subsequences is described herein including at least with respect to FIG. 3A. In some embodiments, processing at least some of the differences at sub-act 224c further includes left normalizing the differences with respect to the reference sequence construct.

In some embodiments, as a result of act 224c, the processed one or more differences may be identified as first variants to be included in the plurality of variants. In the example e of FIG. 3A, insertions “AG” and “A” and deletions “CC” and “G” would be identified as first variants to be included in the plurality of variants. In some embodiments the first variants may be included in one or more input files in any suitable format. For example, the first variants may be included in one or more VCF files. As should be appreciated from the foregoing, sub-acts 224a, 224b, and 224c may be performed for each of at least some of the plurality of alternate sequences obtained at act 222.

Process 220 then proceeds to act 226, where second variants associated with the reference sequence construct are obtained. In some embodiments, the second variants include any variants described with respect to act 202 of FIG. 2A, except for the alternate sequences obtained at act 222.

Process 220 then proceeds to act 228 where merging variants to obtain the plurality of variants is performed. In some embodiments, the obtained plurality of variants includes the plurality of variants that will be used as part of process 200 starting with act 204 (as shown in FIG. 2A, the plurality of variants output from act 202, of which FIG. 2B is showing an example implementation, are provided as input to and are filtered in act 204). In some embodiments, merging the variants includes processing input files that describe the variants and unifying the variant structure for merging. In some embodiments, processing the input files includes splitting multiallelic variants. In some embodiments, processing the input files may include removing non-standard variant definitions, leaving only fully resolved variants. In some embodiments, processing the input files may include additional filters, such as filtering by allele frequency, to choose second variants to be included. For example, in some embodiments, only variants that have an allele frequency of at least a threshold percentage (e.g., at least 2%, at least 5%, at least 10%, at least 15%, etc.) may be included. In some embodiments, processing the input files may further include left normalizing variants. In some embodiments, processing the input files may include clearing unused annotations, clearing certain field (e.g., ID and FILTER fields), and clearing sample information. In some embodiments, processing the input files may include annotating the files with information indicative of allele frequencies. In some embodiments, processing the input files may include annotating variants to indicate a source file (e.g., using an ID assigned to the file.)

After processing the input files, the first and second variants may be merged. In some embodiments, merging the variants includes taking multiple input files and generating a single file (e.g., a VCF file or a file in any other suitable format), which describes an initial graph reference that includes the first and second variants. In some embodiments, merging the input files may include aggregating annotations if a same variant originates from multiple sources. For example, a new effective allele frequency may be calculated for a variant that originates from multiple sources (e.g., having difference allele frequencies and different sample sizes). The final allele frequency may be determined by averaging the original allele frequencies, weighted by the number of samples used for the corresponding source file.

In some embodiments, to perform act 206a of process 200, where identifying a first subset of variants is performed, one or more structural variants are identified for exclusion from the plurality of variants in order to obtain the first subset of variants. FIG. 2C is a flowchart of an example process 240 for identifying one structural variant for exclusion from the plurality of variants. In some embodiments, process 240 can be repeated to identify the one or more additional structural variants for exclusion from the plurality of variants.

As described herein above, the variants obtained at act 202 of process 200 may include structural variants that are (a) large in size and/or (b) include subsequences that are identical to subsequences included elsewhere among the reference sequence construct, other variants, or decoy sequences. As such, process 240 includes filtering such structural variants. An example of filtering structural variants is further described herein including at least with respect to FIG. 3B.

In some embodiments, the process 240 begins at act 242 where determining whether a length of a first structural variant exceeds a specified threshold is performed. In some embodiments, different types of structural variants may be compared to different thresholds. For example, the length of an insertion may be compared to a first threshold (e.g., 2,500 bp, 5,000 bp, 7,500 bp, 10,000 bp, 20,000 bp, etc.), while the length of a deletion may be compared to a second, different threshold (e.g., 50,000 bp, 75,000 bp, 90,000 bp, 100,000 bp, 150,000 bp, 200,000 bp, etc.) In other embodiments, different structural variants may be compared to a same threshold.

Regardless of the threshold, if the length of the first structural variant does exceed the specified threshold, then the structural variant is excluded from the plurality of variants at act 254. If the length of the first structural variant does not exceed the threshold, then the example implementation proceeds to act 244 where determining whether the reference sequence construct includes a subsequence that is identical to a portion of the first structural variant is performed.

In some embodiments, determining whether the reference sequence construct includes a subsequence that is identical to a first portion of the first structural variant includes aligning the structural variant to the reference sequence construct. The reference sequence construct may be compared to the structural variant at the aligned position to determine whether they include any matching subsequences. If the reference sequence construct includes a subsequence that is identical to a subsequence included in the structural variant, then a length is determined for the matching subsequence. In some embodiments, act 244 includes determining whether the length of the matching subsequence is greater than a specified threshold. For example, the specified threshold may be similar to the length of a sequence read (e.g., 50 bp, 100 bp, 150 bp, 200 bp, 250 bp, 300 bp, etc.) In some embodiments, the specified threshold may vary based on the length of one or more sequence reads being aligned. In some embodiments, if the matching subsequence is longer than a sequence read that is to be aligned to a graph reference construct, a sequence read may be inaccurately aligned to both or either subsequence (e.g., included in the structural variant and the reference sequence construct) if the structural variant were to be included in the graph reference construct.

In some embodiments, if the reference sequence construct includes a subsequence that is identical to a portion (e.g., a subsequence) included in the first structural variant, and the length of the subsequence exceeds the specified threshold, then the first structural variant is excluded from the plurality of variants at act 254. If the reference sequence construct does not include a subsequence that is identical to a portion of the first structural variant and has a length that exceeds the specified threshold, then the process 240 proceeds to act 246.

Act 246 may include determining whether a second structural variant includes a subsequence that is identical to a portion of the first structural variant. That determination may be made in any suitable way and, for example, may include aligning the first structural variant to one or more other variants. If a second structural variant includes a subsequence that is identical to a subsequence included in the first structural variant, then a length is determined for the matching subsequence. For example, the specified threshold may be similar to the length of a sequence read (e.g., 50 bp, 100 bp, 150 bp, 200 bp, 250 bp, 300 bp, etc.) In some embodiments, the specified threshold may vary based on the length of one or more sequence reads being aligned. In some embodiments, the threshold may be the same threshold used in act 244. In some embodiments, the threshold may be a different from the threshold used in act 244. In some embodiments, if the matching subsequence is longer than a sequence read that is to be aligned to a graph reference construct, a sequence read may be inaccurately aligned to both or either subsequence (e.g., included in the first structural variant and the second sequence construct) if both the first and second structural variants were included in the graph reference construct.

In some embodiments, if the second structural variant includes a subsequence that is identical to a portion of the first structural variant, and the length of the subsequence exceeds the specified threshold, then process 240 proceeds to act 252. Act 252 may include determining which structural variant to exclude. In some embodiments, it may be desirable to exclude the shorter of the structural variants since the longer structural variant contains more information. As such, act 252 may include determining whether the length of the second structural variant exceeds the length of the first structural variant. Upon determining that the length of the second structural variant exceeds the length of the first structural variant, the first structural variant is excluded from the plurality of variants at act 254. Upon determining that the length of the second structural variant does not exceed the length of the first structural variant, the second structural variant is excluded from the plurality of variants at act 256.

If, at act 246, it is determined that the second structural variant does not include a subsequence that is identical to a portion of the first structural variant and has a length that exceeds the specified threshold, then the process 240 proceeds to act 248. Act 248 may include determining whether a decoy sequence includes a subsequence that is identical to a portion of the first structural variant. As described herein, a decoy sequence may include common sequences that are not included in the reference. However, if one of the common sequences is already represented by a structural variant, then there is no need to include that sequence as a decoy. Therefore, if a decoy sequence includes a subsequence that is identical to a subsequence included in the first structural variant, then that region of the decoy sequence is masked at act 258. The process 240 then proceeds to act 250 where the first structural variant is included in the first subset of variants.

FIG. 2D is a flowchart illustrating a process 260 for identifying the filtered set of variants from among the first subset of variants, according to some embodiments of the technology described herein. Process 260 is an example for how act 206b of process 200 may be implemented.

As described herein above, as more variants are included in a graph reference construct, it becomes more likely that identical paths are included in different regions of the graph reference construct. Aligning sequence reads to such a graph reference construct may result in ambiguous and uninformative results, due to multi-mapped sequence reads. In some embodiments, the quality of alignment is indicative of a probability that the alignment is correct. The mapping quality may be low if there are multiple regions in the graph to which a sequence read is mapped (e.g., multi-mapped). In some embodiments, a filtering stage, such as example implementation 206b, may be used to exclude some variants that result in multi-mapped sequence reads (e.g., multiply-alignable variants) to break the identity of different regions in the graph reference construct. An example of filtering multiply-alignable variants is described herein including at least with respect to FIG. 3C.

In some embodiments, the example implementation 206b begins at 262 where generating an initial graph reference construct is performed using the reference sequence construct and at least some variants of the first subset of variants identified at act 250 of process 240. In some embodiments, at least some variants in the first subset of variants may be added to the reference sequence construct using one or more nodes and/or edges to generate the initial graph reference construct. Therefore, the initial graph reference construct may include one path representing the reference sequence construct and one or more paths representing variants included in the initial graph reference construct. An “edge combination” may refer to a path in the initial graph reference construct that follows one or more particular edges, and therefore, represents one or more variants associated with those edges (e.g., the variant is included as the edge, included as node that follows the edge, etc.).

The example implementation 206b then proceeds to act 264 where the initial graph reference construct is traversed to generate, synthetically from the graph reference construct, a plurality of graph reads of a specified length. A graph read may include one or more nucleotides that represent a path at a certain region in the initial graph reference construct. In some embodiments, the graph reads are generated for all possible haplotypes in the graph. In some embodiments, traversing the initial graph reference construct to generate the graph reads includes traversing the graph reference construct using a sliding window with a skip. In some embodiments, act 264 includes performing sub-acts 264a and 264b.

Sub-act 264a, in some embodiments, includes generating a first subset of graph reads by traversing the graph reference construct over a first interval. In some embodiments, the first subset of graph reads may include one reference graph read and one or more non-reference graph reads. The reference graph read may represent the path through the reference sequence construct, while the non-reference graph reads may represent paths that follow edges (e.g., edge combinations) in the initial graph reference construct in that interval.

Sub-act 264b may include generating a second subset of graph reads by traversing the initial graph reference construct over a second interval that partially overlaps the first interval. As described herein above, the second subset of graph reads may include one reference graph read and one or more non-reference graph reads. In some embodiments, since the first and second intervals overlap, graph reads included in the second subset of graph reads may represent one or more variants that are represented by graph reads included in the first subset of graph reads.

In some embodiments, after the plurality of graph reads are generated at act 264, example implementation 206b proceeds to act 266, where the plurality of graph reads is aligned to the initial graph reference construct to determine qualities of alignment for each of at least some of the plurality of graph reads. As described herein above, a quality of alignment may be indicative of the probability that a graph read is correctly aligned to the initial graph reference construct. Determining a quality of alignment for a graph read may include determining whether a graph read maps to more than one region in the initial graph reference construct. In some embodiments, a graph read that maps to more than one region in the initial graph reference construct results in a lower quality of alignment than a graph read that maps to just one position in the graph reference. This is because a graph read that maps to only one position in the initial graph reference construct is more likely to be mapped to the correct position than a graph read that may map to multiple positions.

In some embodiments, for a subset of graph reads (e.g., the first subset of graph reads, or the second subset of graph reads), the quality of alignment determined for the reference graph read is compared to the qualities of alignment determined for the non-reference graph reads. In some embodiments, if a non-reference graph read has a quality of alignment that is less than the quality of alignment determined for the reference graph read, then the edge combination represented by the non-reference graph read may be identified for exclusion from the filtered set of variants. For example, if the quality of alignment of a non-reference graph read is zero, while the quality of alignment of the reference graph read is greater than 0, then the edge combination represented by the non-reference graph read is identified for exclusion from the filtered set of variants. In some embodiments, if a quality of alignment determined for a non-reference graph read is greater than the quality of alignment determined for a reference graph read, then the edge combination represented by the non-reference graph read may be identified for inclusion in the filtered set of variants. Additionally or alternatively, if a non-reference graph read has a quality of alignment that is greater than a specified threshold (e.g., at least 10, at least 20, at least 30, at least 40, etc.), the edge combination represented by the non-reference graph read may be identified for inclusion in the filtered set of variants. It should be appreciated, however, that while an edge combination may be identified for inclusion or exclusion from the filtered set of variants, in some embodiments, the edge combination may not actually be included or excluded from the filtered set of variants at act 268 of process 200.

After qualities of alignment are determined for at least some of the plurality of graph reads, example implementation 206b proceeds to act 268 where at least some of the first subset of variants are excluded from a filtered set of variants. In some embodiments, at act 268, non-reference graph reads which include a same edge combination may be grouped. For example, since the first and second intervals overlap, a non-reference graph read included in the first subset may represent an edge combination that is also represented by a non-reference graph read included in the second subset. As such, those graph reads may be grouped.

In some embodiments, if each of the grouped non-reference graph reads was identified for exclusion from the filtered set of variants at act 266, this may indicate that the edge combination results in multi-mapped sequence reads (e.g., reads that align to multiple different regions of the graph). Therefore, the edge combination may be identified for filtration. In some embodiments, a set of variants represented by an edge combination identified for filtration is excluded from the filtered set of variants at act 268. For example, a set of variants is identified from the edge combinations identified for filtration such that each edge combination has at least one variant excluded from the filtered set of variants.

FIG. 3A is a diagram of an illustrative example of processing alternate sequences associated with a reference construct, according to some embodiments of the technology described herein. The example of FIG. 3A serves as an example of performing act 224 of process 220.

In some embodiments, example 300 begins at act 302 where aligning an alternate sequence to the reference sequence construct is performed. As part of the alignment, one or more differences are identified at the aligned position, represented by the shaded boxes. The example includes annotations that identify regions that match and regions that include structural variants, along with the number of nucleotides included in that region. In some embodiments, a region that is annotated with “M” is indicative of matching nucleotides. For example, the region annotated with “M3” represents three matching nucleotides, while the region annotated with “M23” represents 23 matching nucleotides. In some embodiments, a matching region may include one or more mismatches. For example, the region “M23” includes two mismatches. First, at position 19, the nucleotide “G” in the reference sequence construct does not match nucleotide “T” in the alternate sequence. Second, at position 30, the nucleotide “G” in the reference sequence construct does not match the nucleotide “T” in the alternate sequence. In some embodiments, a region that is annotated with “I” is indicative of an insertion. For example, the region “I5” represents an insertion of five nucleotides. As shown in the alternate sequence, five shaded boxes represent the insertion of nucleotides “GACCG.” As another example, the region “I4” represents an insertion of four nucleotides. As shown in the alternate sequence, four shaded boxes represent the insertion of nucleotides “AGTT.” In some embodiments, a region that is annotated with “D” is indicative of a deletion. For example, the region “D4” represents a deletion of four nucleotides. As shown in the reference sequence construct, four shaded boxes represent the deletion of nucleotides “TACC.” As another example, the region “D3” represents a deletion of three nucleotides. As shown in the reference sequence construct, three shaded boxes represent the deletion of nucleotides “AAT.”

In some embodiments, some of the differences identified at act 302 may be processed at act 304. In some embodiments, act 304 may include splitting complex variants, such as insertion and deletion events, to generate smaller variants. For example, the consecutive insertion and deletion events, represented by regions “I5” and “D4,” may be processed at act 304. As shown, the insertion and deletion events are aligned against one another to determine whether they include any matching nucleotides. The aligned position includes a matching region “M4” and an insertion region “I1.” The matching region includes one mismatch, as represented by the grey boxes, and three matches. Therefore, the complex variants (e.g., the insertion and deletion events) can be split into smaller variants. As shown, the mismatching nucleotides in the region “M4” can be represented as a single nucleotide polymorphism (SNP), while the insertion in the region “I1” can be represented by a single nucleotide insertion. The matching region is excluded, which (a) simplifies the variants and (b) reduces the size of the variants.

First variants are obtained at act 306, which represent simplified versions of the alternate sequences. As shown, the variants are left normalized, meaning the start position of the variant is shifted the left. In some embodiments, the first variants may be annotated to indicate the start position with respect to the reference sequence construct. For example, the first variant annotated with the number “4” indicates that it starts at the fourth position (e.g., fourth nucleotide) from the left of the reference sequence construct.

In some embodiments, a VCF file may be output that includes the first variants obtained at act 306. The VCF file may include the position of the variant and the nucleotides that define the variant with respect to the reference sequence construct and the alternate sequence. For example, at position 13, the alternate sequence includes nucleotide “C,” which matches the first nucleotide in the sequence “CAAT,” of the reference sequence construct. The nucleotides “AAT” represent a deletion event that follows the nucleotide at position 13 in the alternate sequence. Therefore, the reference sequence includes the nucleotides “AAT” following position 13, but the alternate sequence does not.

FIG. 3B is a diagram of an illustrative example of performing a first stage of a multi-stage variant filtering technique, with the first stage being used to identify a set of structural variants to be excluded from an initial set of variants, according to some embodiments of the technology described herein. The example of FIG. 3B serves as an example of performing act 206a of process 200, as described herein including at least with respect to FIG. 2A, where one or more structural variants are identified for exclusion from a plurality of variants.

In this example, identifying the set of structural variants includes four stages 322, 324, 326, and 328. However, it should be appreciated that in some embodiments, one or more stages may be omitted. For example, if no decoy sequences are associated with the reference sequence construct, then stage 328 may be omitted. Such an omission would not affect the performance of the remaining three stages 322, 324, and 326.

In some embodiments, the first stage 322 includes aligning structural variants, such as insertions, to the reference sequence construct. As shown in FIG. 3B, two structural variants are aligned to the reference sequence construct to determine two alignments, alignment 332 and alignment 334.

In some embodiments, at the aligned position, the first structural variant is compared to the reference sequence construct to determine whether the first structural variant includes a subsequence that is identical to a subsequence included in the reference sequence construct and has a length that is greater than a specified threshold. In other words, this may include determining the length of matching regions at the aligned position. For example, when the first structural variant is aligned to the reference sequence construct to determine alignment 332, it includes three matching regions. The first matching region has a length of eight nucleotides, the second matching region has a length of 42 nucleotides, and the third matching region has a length of 19 nucleotides. Compared to an example threshold of 30 nucleotides, the length of the second matching region (e.g., 42 nucleotides) exceeds the threshold. Therefore, the first structural variant would be excluded from the plurality of variants.

In some embodiments, if the first structural variant was included in the plurality of variants and used to generate the graph reference construct, rather than being filtered out at act 322, it may have resulted in ambiguous sequence read alignment. For example, a sequence read that has a length of less than 42 nucleotides (e.g., 30 nucleotides) may align to both the reference sequence construct and the first structural variant within the matching region. In this case, there would be no way to determine which alignment is correct, causing the alignment to be uninformative.

As another example, when the second structural variant is aligned to the reference sequence construct to determine alignment 334, it includes four matching regions. The first matching region has a length of eight nucleotides, the second matching region has a length of 20 nucleotides, the third matching region has a length of 18 nucleotides, and the fourth matching region has a length of 19 nucleotides. Since none of the matching regions have a length that exceeds the example threshold of 30 nucleotides, the second structural variant is not excluded from the plurality of variants.

In some embodiments, a second stage 324 includes filtering structural variants based on their size. For example, if the length of a deletion event is greater than a maximum deletion size threshold (e.g., 90,000 bp), then the deletion event may be excluded from the plurality of variants. Similarly, if the length of an insertion event is greater than a maximum insertion size threshold (e.g., 5,000 bp), then the insertion event may be excluded from the plurality of variants. If the lengths of the insertion or deletion events do not exceed the maximum size thresholds, then those structural variants may be included in the plurality of variants or undergo further filtering stages 322, 326, 328. In some embodiments, structural variants which have been excluded from the plurality of variants may be included as additional decoy sequences.

In some embodiments, the third stage 326 includes determining whether the two structural variants include an identical subsequence of a length that exceeds a specified threshold. As shown in the first alignment 338, there are two matching regions (e.g., two identical subsequences). The first matching region has a length of 8 nucleotides, while the second matching region has a length of 51 nucleotides. Since the length of the second matching region (e.g., 51 nucleotides) exceeds the example threshold of 30 nucleotides, one of the structural variants is excluded from the plurality of variants. Since the longer structural variant contains more information, the shorter structural variant is excluded from the plurality of variants.

As another example of stage 326, alignment 340 shows the alignment of a different pair of structural variants. As shown, the alignment 340 includes three matching regions. The first matching region has a length of 6 nucleotides, the second matching region has a length of 22 nucleotides, and the third matching region has a length of 326 nucleotides. Since none of the matching regions have a length that exceeds the example threshold of 30 nucleotides, neither structural variant is excluded from the plurality of variants.

In some embodiments, filtering stage 328 includes aligning a structural variant to a decoy sequence to obtain aligned position 342. Since the sequence represented by the structural variant will be included in the graph reference construct, there is no reason to additionally include it in the decoy sequences. Further, including the sequence in the decoy sequence would cause a sequence read to align to both the decoy sequence and the structural variant representing that sequence. Therefore, the decoy sequence is masked at the aligned position to obtain a masked decoy sequence 344. In some embodiments, the structural variant may not align to a decoy sequence at filtering stage 328. Therefore, no region of a decoy sequence would be masked.

In some embodiments, illustrative example 320 is done as part of act 206a and, in this example, the structural variants that are not excluded from the plurality of variants would have been included as part of the first subset of variants created in process 206a.

FIG. 3C is a diagram of an illustrative example of performing a second stage of a multi-stage variant filtering technique, with the second stage being used to identify a set of multiply-alignable variants to be excluded from the initial set of variants, according to some embodiments of the technology described herein. The example of FIG. 3C serves as an example of performing act 206b of process 200, where one or more multiply-alignable variants are identified for exclusion from a plurality of variants.

In some embodiments, an initial graph reference construct 362 may be generated. In some embodiments, this may include adding the first subset of variants, obtained as a result of using the first filtering stage (e.g., the first filtering stage described herein including at least with respect to FIGS. 2A and 3B), to a reference sequence construct. As shown in the example, the initial graph reference construct includes a variant at position 12, a variant at position 16, and a variant beginning at position 36. The variants are represented in the graph using nodes and edges.

In some embodiments, a first stage 352 includes generating a plurality of graph reads by traversing the initial graph reference 362 over specified intervals. As shown, a first subset of graph reads 364 are generated for a first interval in the graph. The first subset of graph reads 364 includes one graph read that represents a path through the reference sequence construct, represented by the graph read that includes only white squares. The remaining graph reads included in the first subset of graph reads 364 represent paths that include different combinations of edges in the graph. For example, one graph read represents a path that continues along the edge at position 12, including the variant represented at that position. Another graph read represents a path that continues along the edge at position 16. The final graph read represents a path that continues along both edges, the edge at position 12 and the edge at position 16.

The second subset of graph reads 366 are generated by traversing the initial graph reference construct over a second interval in the initial graph reference construct that overlaps the first interval. Similarly, the second subset of graph reads 366 include a reference graph read, and three non-reference graph reads representing the three different edge combinations (e.g., the edge at position 12, the edge at position 16, and the edges at positions 12 and 16). As shown, overlapping intervals result in graph reads that include the same edge combinations.

Finally, the third subset of graph reads 368 are generated by traversing the initial graph reference construct over a third interval in the initial graph reference construct that overlaps the second interval. The third interval only includes one edge combination, as represented by the variant included at position 36. Therefore, the third subset of graph reads 368 includes one reference graph read and one non-reference graph read.

In some embodiments, the generated plurality of graph reads may be collected as a FASTQ file. As shown at stage 354, using the FASTQ file and the initial graph reference construct 362, the plurality of graph reads may be aligned against the initial graph reference construct using a graph aligner to obtain a BAM file, used to represent aligned sequences. In some embodiments, the BAM file may include a quality of alignment, or mapping quality (“MQ”), for each of the graph reads. The quality of alignment may be indicative of the probability of correct alignment. As described herein above, including with respect to FIG. 2D, a graph read that has a low quality of alignment may indicate that the graph read aligns to more than one location in the initial graph reference construct.

Shown at stage 356, each graph read is annotated with a quality of alignment (“MQ”). If the quality of alignment of a non-reference graph read is less than the quality of alignment of the reference graph read, then the edge combination represented by the non-reference graph read is identified for exclusion from the filtered set of graph reads (labelled as “bad” in FIG. 3C). If the quality of alignment of a non-reference graph read is greater than the quality of alignment of the reference graph read, and/or is greater than a specified threshold, then the edge combination represented by the graph read is identified for inclusion in the filtered set of graph reads (labelled as “good” in FIG. 3C). Otherwise, the edge combination and associated graph reads are ignored.

As shown in the example, the first subset of graph reads includes a reference graph read with a quality of alignment of 25. Since each of the non-reference graph reads have qualities of alignments that are less than 25 (e.g., 0), the edge combinations represented by the non-reference graph reads are identified for exclusion form the filtered set of graph reads. The second subset of graph reads 374 includes a reference graph read with a quality of alignment of 35. Since two of the non-reference graph reads have qualities of alignments that are less than 35, the edge combinations represented by those graph reads are identified for exclusion from the filtered set of graph reads. One non-reference graph read included in the second subset 374 has a quality of alignment of 45, which is greater than the quality of alignment of the reference graph read. Therefore, the edge combination represented by that graph read is identified for inclusion in the filtered set of graph reads. Finally, since both the reference and non-reference graph reads of the third subset 376 have the same mapping quality of 0, this subset 376 is ignored.

After classification, the graph reads are grouped by the edge combinations that they represent. For example, the first group 378 represents the edge combination that includes the variant “G” at position 16, the second group 380 represents the edge combination that includes the variant “T” at position 12, and the third group 382 represents the edge combination that includes both variants “T” and “G” at positions 12 and 16 respectively.

Each group 378, 380, 382 is then classified based on the classifications of the graph reads included in the group. For example, group 378 includes graph reads that were all identified for exclusion from the filtered set of variants. This indicates that the edge combination that includes the variant “G” may result in identical paths at different regions in the initial graph reference construct 362, leading to multi-mapped sequence reads. Therefore, the group 378 is identified for filtration. Group 380 includes mixed classifications (e.g., graph reads identified for both inclusion and exclusion from the filtered set of variants.) Therefore, group 380 is not identified for filtration. Finally, group 382 includes graph reads that are all identified for exclusion from the filtered set of variants. Therefore, group 382 is identified for filtration.

After the groups are classified, a set of variants from among the variants included in the groups identified for filtration are excluded from the first subset of variants. Identifying the set of variants, in some embodiments, includes identifying one or more variants that are common to groups identified for filtration. For example, as shown in FIG. 3C, the variant at position 16 is identified for exclusion because it is included in both groups 378, 382 identified for filtering. Therefore, that variant is excluded from the first subset of variants.

In some embodiments, illustrative example 350 is done as part of act 206b and, in this example, the variants that are not excluded from the plurality of variants would have been included as part of the filtered of variants created in process 206b.

In some embodiments, the filtered set of variants are used to generate the graph reference construct at stage 384.

### Further Aspects of Graph Construction

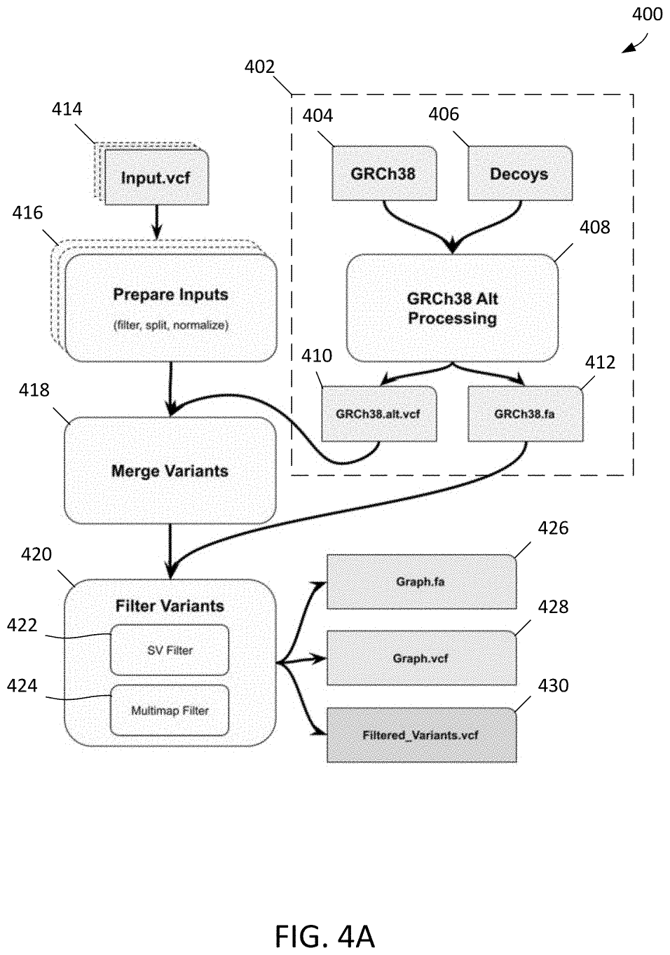

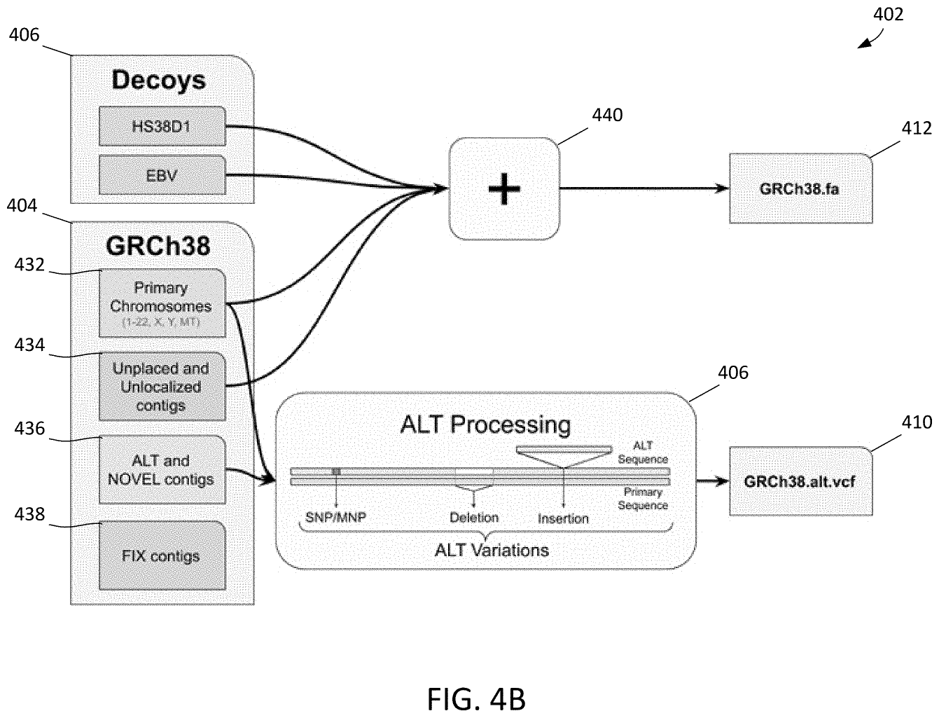

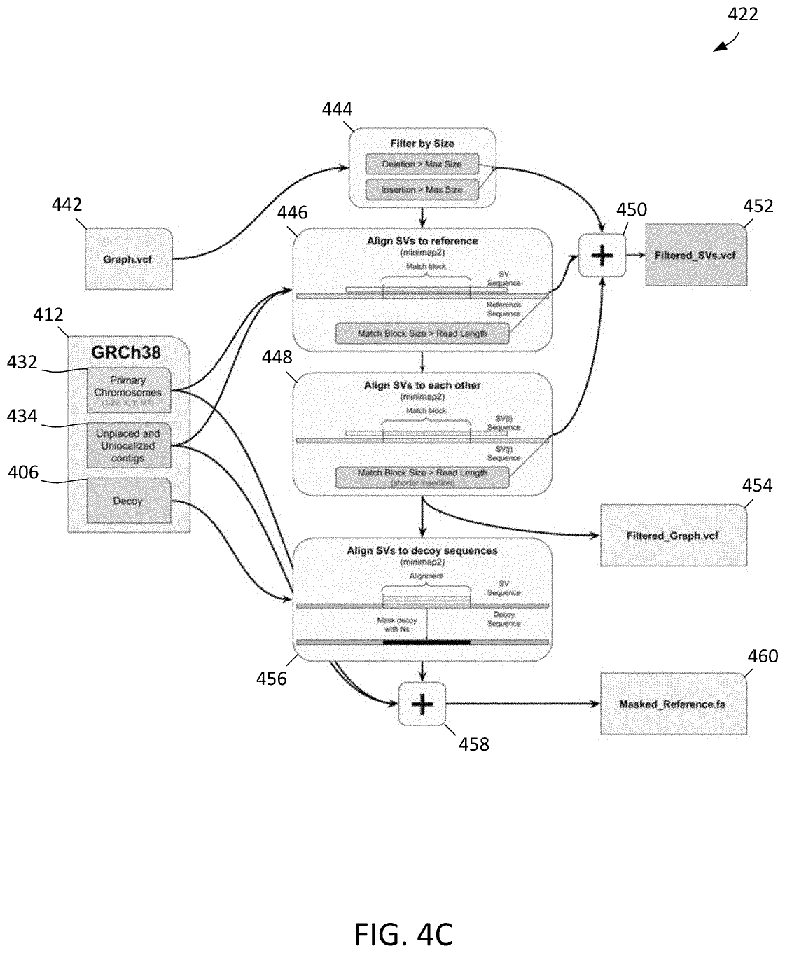

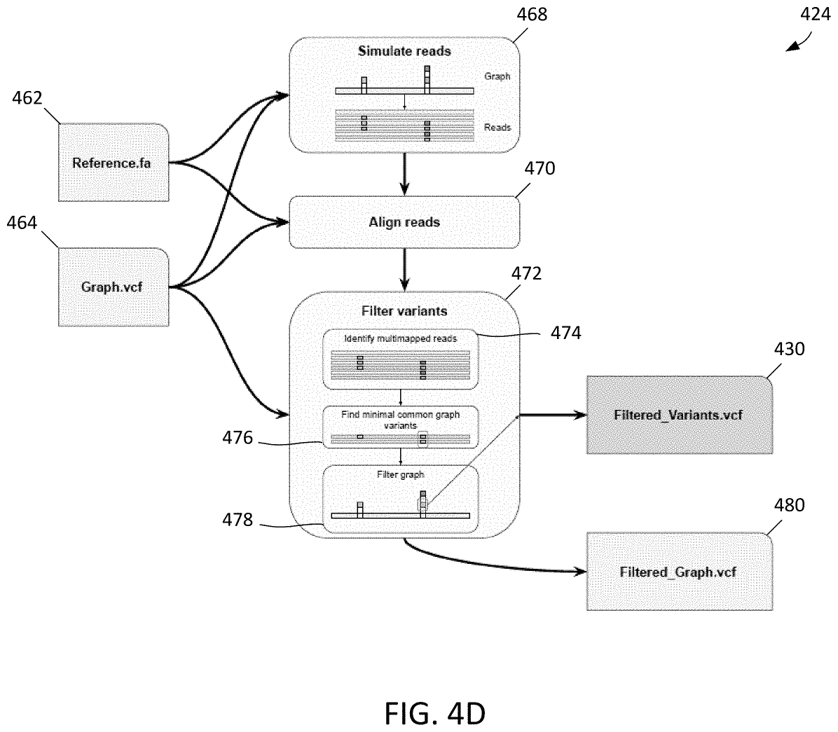

Additional aspects of the graph construction techniques described herein are described below with reference to FIGS. 4-8.

FIG. 4A is a diagram depicting an exemplary process 400 for generating a graph reference construct, according to some embodiments of the technology described herein. In some embodiments, process 400 is an example implementation of illustrative technique 100 and process 200 for generating a graph reference construct, described herein including at least with respect to FIGS. 1 and 2A.

In some embodiments, process 400 includes act 408, where a linear reference construct is processed. In some embodiments, prior to act 408, the process 400 includes obtaining a linear reference construct 404 and decoys 406 associated with the linear reference construct 404. For example, as shown in FIG. 4B, the linear reference construct 404 in this example is the GRCh38 genome assembly. In some embodiments, the GRCh38 genome assembly includes primary chromosomes 432, unplaced and unlocalized contigs 434, alternate (ALT) contigs and NOVEL contigs 436, and FIX contigs 438.

In some embodiments, ALT and NOVEL contigs 436 represent alternate sequences for certain regions in the canonical chromosomes. These regions show high variability in the population and the ALT and NOVEL contigs 436 are provided as additional sequences to augment the haploid genome. In some embodiments, the ALT and NOVEL contigs 436 are obtained as General Feature Format (GFF) files. The GFF files describe the alignment of the alternate contigs with respect to the canonical regions in Concise Idiosyncratic Gapped Alignment Report (CIGAR) format. Though it should be appreciated that the data may be formatted in any other suitable format, as aspects of the technology described herein are not limited in this respect. In some embodiments, the ALT contigs are examples of alternative sequences, described herein including at least with respect to FIGS. 1A-3C.

In some embodiments, act 408 includes processing the linear reference construct 404 to remove the ALT and NOVEL contigs 436 from the linear reference 404 so that it only contains primary chromosomes and unplaced and unlocalized contigs 434. Additionally or alternatively, decoys 406 may be added to the linear reference construct, at act 408, to obtain linear reference construct 412. The linear reference construct 412 may be output as a FASTA file or data in any other suitable format.

In some embodiments, after the ALT and NOVEL contigs 436 are removed from the linear reference 404, they are mapped to the primary chromosomes 432. Since the ALT and NOVEL contigs 436 often contain long stretches of sequences that are identical to the linear reference, further processing is performed at act 408 to (a) decompose the ALT and NOVEL contigs 436 into smaller variants and to (b) left normalize those decomposed variants. The resulting variants 410 may be output as a FASTA file or data in any other suitable format. Act 408 is an example of the type of processing that may be performed in act 224 of process 220 where processing alternative sequences to obtain second variants associated with the reference sequence construct is performed, as described herein including at least with respect to FIG. 2B.

As one example of decomposing the ALT and NOVEL contigs 436, consecutive insertion and deletion events can be combined and the alignment can be simplified (e.g., simplified into a number of SNPs). In some embodiments, in order to obtain a more minimal representation of the variations, the variations are aligned to each other using Needleman-Wunsch algorithm, for example. If a long sequence of identical matching blocks is identified in the alignment, then this variation may be split into smaller variations from the matching blocks.

In some embodiments, process 400 includes act 416 where inputs 414 are obtained and prepared. In some embodiments, the inputs 414 include variant files (e.g., VCF) files. In some embodiments, the inputs 414 are obtained from one or multiple sources. In the case that the inputs 414 are obtained from different sources, preparing the inputs at act 416 includes processing the inputs 414 to unify the variant structure. For example, processing the inputs 414 may include: splitting multiallelic variants, removing non-standard variant definitions and leaving only fully sequence resolved variants, filtering by allele frequency, left normalizing the variants, clearing unused annotations, clearing ID and FILTER fields, clearing sample information, annotating with information used to calculate effective allele frequency, and/or annotating variants to indicate the original source file using the ID assigned to the respective VCF file.

Act 408 and act 414 may be performed in any order. In some embodiments, act 408 and act 414 are performed contemporaneously.

After obtaining and preparing the inputs 414 at act 416 and processing the linear reference construct 404 and decoys 406 at act 408, process 400 proceeds to act 418, where the variants are merged. Act 418 is an example of the type of processing that may be performed in act 228 of process 220 where merging the first and second variants to obtain the plurality of variants is performed, as described herein including at least with respect to FIG. 2B. In some embodiments, merging variants at act 418 includes processing multiple input variant files to obtain a single biallelic candidate graph file. For example, act 418 may include processing the prepared inputs 414 and the alternate variants 410 to obtain a single output file describing an initial graph reference construct. In some embodiments, the merging includes combining all variants into a single set. If the same variant originates from multiple sources, then merging at act 418 includes aggregating annotations associated with the variant and calculating an effective allele frequency for the variant. In some embodiments, the effective allele frequency for a variant is the average of allele frequencies originating from all of the source files, weighted by the number of samples used for the corresponding source file.

Process 400 then proceeds to act 420 where the variants (e.g., the variants output at act 418) are filtered. In some embodiments, the variants may be filtered at act 420 using a multi-stage filtering technique to identify a set of variants 430 that should be excluded from a graph reference construct 426, 428 obtained as output of process 400. Act 420 is an example of the type of processing that may be performed in act 206 of process 200 where filtering a plurality of variants associated with a reference sequence construct is performed to obtain a filtered set of variants, as described herein including at least with respect to 2A.

In some embodiments, filtering at act 420 includes a structural variant (SV) filter 422 and a multimap filter 424. In some embodiments, the SV filter 422 is a type of filter that can be used as part the first filtering stage 206a of process 200, described herein including at least with respect to FIG. 2A. In some embodiments, the SV filter 422 may be used to identify structural variants that are to be excluded from the graph reference construct 426, 428. This may eliminate introducing sequences that would result in duplications in the graph reference construct. An example of using the SV filter 422 is described herein including at least with respect to FIG. 4C.

In some embodiments, the multimap filter 424 may be used as part of the second filtering state 206b of process 200, described herein including at least with respect to FIG. 2A. In some embodiments, the multimap filter 424 may be used to identify multiply-alignable variants that, if included in the graph reference construct 426, 428, would cause sequence reads to align to multiple regions of the graph reference construct 426, 428 (e.g., resulting in multimapping problems). In some embodiments, the identified variants are excluded from the graph reference construct 426, 428. An example of using the multimap filter 424 is described herein including at least with respect to FIG. 4D.

In some embodiments, filtered variants 430 and graph reference construct 426, 428 are obtained as an output of filtering at act 420. In some embodiments, the filtered variants 430 include those variants identified for exclusion using the SV filter 422 and the multimap filter 424. The filtered variants 430 may be output as a VCF file or as data in any other suitable format.

In some embodiments, the graph reference construct 426, 428 is obtained by aligning variants excluded from the filtered variants 430 against the linear reference construct 412 output at act 408. For example, the variants include the variants output at act 418 that were not identified for exclusion at act 420. In some embodiments, the graph reference construct is output as a FASTA file 426, as a VCF file 428, and/or as data in any suitable format.

FIG. 4C is a diagram depicting an exemplary process 422 for identifying a set of structural variants, according to some embodiments of the technology described herein. In some embodiments, the process 422 includes processing structural variants represented in an initial graph reference construct 442 (e.g., output at act 418, where merging is performed).

In some embodiments, act 444 includes filtering the structural variants by size. For example, structural variants with sizes that exceed a threshold may be excluded from the filtered graph 454 and included in the filtered structural variants 452. In some embodiments, insertions are compared to a different threshold than deletions, or insertions and deletions are compared to the same threshold.

In some embodiments, act 446 includes aligning the structural variants that were not filtered out at act 444 to the linear reference construct 412. The structural variants may be aligned using any suitable alignment technique, such as, for example, the Minimap2 technique, described by Heng Li (“Minimap2: pairwise alignment for nucleotide sequences”, Bioinformatics, Vol. 34, Issue 18, 2018, pp. 3094-3100), which is incorporated by reference herein in its entirety. If there is a subsequence (e.g., a match block) in the structural variant that is identical to the non-decoy sequences in the linear reference and is at least the length of a sequence read (e.g., 150 bp), then the structural variant is excluded from the filtered graph 454 and included in the filtered structural variants 452. In some embodiments, the threshold is chosen to be the length of a sequence read, since it is difficult for variant callers to reassemble the sequence reads to detect structural variants when there is an alignment gap that is larger than the sequence read length.

In some embodiments, act 448 includes aligning the structural variants that were not filtered out at act 446 to each other. The structural variants may be aligned using any suitable alignment technique, such as, for example, Minimap2. If there is a common identical subsequence of at least read length, then the smaller of the structural variants is excluded from the filtered graph 454 and included in the filtered structural variants 452.

In some embodiments, the structural variants that are not filtered out at act 448 are included in the graph reference construct 454. However, since decoys for a reference are obtained by common additional sequences that are not in the linear reference, it is possible that some of those sequences are already represented by the structural variants included in the graph reference construct 454. Therefore, in some embodiments, the structural variants are aligned to decoy sequences at act 456. If there are alignments found, those regions in the decoy are masked with the corresponding number of bases. In some embodiments, the masked decoy sequences are joined with the linear reference sequence 412 at act 458 to generate a masked reference 460, which may be output as a FASTA file or data in any other suitable format.

FIG. 4D is a diagram depicting an exemplary process 424 for using a second filtering stage to identify a set of multiply-alignable variants, according to some embodiments of the technology described herein. In some embodiments, the process 424 includes processing variants represented in an initial graph reference construct 462. In some embodiments, the initial graph reference construct 462 is the same as graph reference construct 442 (e.g., output at act 418, where merging is performed). In some embodiments, the initial graph reference construct 464 is the same as the filtered graph reference construct 454 output from process 422.

In some embodiments, act 468 includes simulating reads that traverse all possible paths in the graph reference construct 464. This includes, for example, traversing the graph reference construct 464 at specified intervals for start positions. For a given start position, all possible paths of a specified length are generated as reads for that position. In some embodiments, the generated reads are collected as a FASTQ file or data in any other suitable format.

In some embodiments, act 470 includes aligning the reads against the graph reference 464 using any suitable alignment technique.

In some embodiments, act 472 includes filtering the variants based on the alignments. In some embodiments, filtering the variants includes grouping reads at the same start position. Within the group, there will be one read that corresponds to the linear reference 462 only, and the rest will follow possible combinations of edges in the graph construct 464.

After the reads are grouped, in some embodiments, non-reference reads will be identified for exclusion from the filtered graph reference construct 480 (e.g., classified as “BAD”). A read is classified as BAD if it has a mapping quality of zero where the reference read had a mapping quality greater than zero. In some embodiments, non-reference reads will be identified for inclusion in the filtered graph reference construct 480 (e.g., classified as “GOOD”) if the read has a mapping quality that is greater than the mapping quality of the reference read or greater than a threshold (e.g., 20). In some embodiments, non-reference reads will not be ignored if they do not meet the above-specified criteria.

In some embodiments, if there are reads that have different starting positions, but follow the same combination of edges, those reads are aggregated. If the aggregated group of reads only includes reads that were classified as “BAD”, then the edge combination is identified for filtration (e.g., flagged).

In some embodiments, at act 476, a minimal subset of edges is identified from the edge combinations that were identified for filtration. For example, the minimal subset of edges may be identified such that each flagged edge combination will have at least one common edge with the subset.

In some embodiments, at act 478, the variants associated with the subset of edges are included in the filtered set of variants 430 and excluded from the filtered graph construct 480.

### Illustrative Example

An experiment was conducted to evaluate the performance of graph reference constructs obtained using the techniques described herein. The experiments show that a graph construct can significantly improve both read alignment and variant calling accuracy while being computationally efficient. The results were compared with those obtained using conventional linear non-graph-based techniques and clearly show that a graph-based approach achieves significantly lower read mapping errors, increased variant calling sensitivity, and provide the improvements of joint variant calling without the computationally intensive post-processing steps. The conventional techniques align sequence reads against a linear reference using BWA-MEM and then identify differences in the data with respect to the linear reference (variant calling) using GATK. The conventional techniques are referred to herein as “BWA+GATK”. BWA-MEM is described by Li H. and Durbin R. (“Fast and accurate short read alignment with Burrows-Wheeler Transform”. Bioinformatics, 25:1754-60, 2009). GATK is described by McKenna A, et al., (“The Genome Analysis Toolkit: a MapReduce framework for analyzing next-generation DNA sequencing data. Genome Res”, 20:1297-303, 2010), which is incorporated by reference herein in its entirety.

To demonstrate the capabilities of the technology described herein, one pan-genome graph and six population-specific graphs were generated according to the techniques described herein, including at least with respect to FIGS. 1-4D. Public databases, such as gnomAD and UK BioBank were used to construct the pan-genome graph. Similarly, public databases were used to construct an initial population-specific graph. The initial population-specific graph was then iteratively augmented with construction sets comprising Illumina sequencing data of the African samples from the 1000 Genomes Project. Pan-African 0 refers to the population-specific graph obtained using only gnomAD and Pan-African 5 is the final graph obtained after all five construction sets have been added to the graph. In the construction of Pan-African 1 graph, the high-quality SVs curated by the Human Genome Structural Variation Consortium (HGSVC) using PacBio HiFi sequencing data for 10 African samples in the 1000 Genomes dataset are also incorporated. The graph and index memory usage and the total number of variants in each graph are listed in Table 1. The contents of each graph are shown in Table 2.

TABLE 1
Size of pan-genome and population-specific graphs

| | Graph and Index | Total Number |
|---|---|---|
| Graph Name | Memory Usage | of Variants |
| Pan-Genome | 16.70 GB | — |
| Pan-African 0 | 16.01 GB | 12.1M |
| Pan-African 1 | 16.43 GB | 12.9M |
| Pan-African 2 | 16.71 GB | 13.0M |
| Pan-African 3 | 16.69 GB | 13.1M |
| Pan-African 4 | 16.73 GB | 13.2M |
| Pan-African 5 | 16.85 GB | 13.2M |

TABLE 2
Contents of pan-genome and population specific graphs.
Other type includes complex insertions and deletions.

| | TOTAL EDGES | | | | | |
|---|---|---|---|---|---|---|
| Workflow | SNP | MNP | INS | DEL | Other | Total |
| Pan-Genome | 1253627 | 10659 | 821544 | 1133352 | 3006 | 14504832 |
| Pan-African 0 | 9278644 | 10655 | 1404792 | 1492475 | 2957 | 12189523 |
| Pan-African 1 | 10583590 | 10144 | 1674791 | 1767556 | 2735 | 14038816 |
| Pan-African 2 | 11158974 | 10140 | 1763463 | 1843941 | 2728 | 14779246 |
| Pan-African 3 | 11522421 | 10142 | 1820040 | 1891888 | 2726 | 15247217 |
| Pan-African 4 | 11777384 | 10145 | 1860915 | 1926501 | 2728 | 15577673 |
| Pan-African 5 | 11967428 | 10141 | 1892349 | 1952936 | 2727 | 15825581 |
| | USED EDGES | | | | | |
| Workflow | SNP | MNP | INS | DEL | Other | Total |
| Pan-Genome | 4355793 | 7583 | 636164 | 393526 | 2074 | 5395140 |
| Pan-African 0 | 4345684 | 7490 | 1184396 | 73381 | 1940 | 6273391 |
| Pan-African 1 | 4862675 | 6935 | 1372664 | 845054 | 1642 | 7088971 |
| Pan-African 2 | 5018916 | 6920 | 1433605 | 867760 | 1628 | 7328839 |
| Pan-African 3 | 5108480 | 6925 | 1472150 | 881391 | 1623 | 7470570 |
| Pan-African 4 | 5167509 | 6925 | 1499816 | 890976 | 1623 | 7566848 |
| Pan-African 5 | 5209844 | 6918 | 1520818 | 898220 | 1619 | 7637419 |

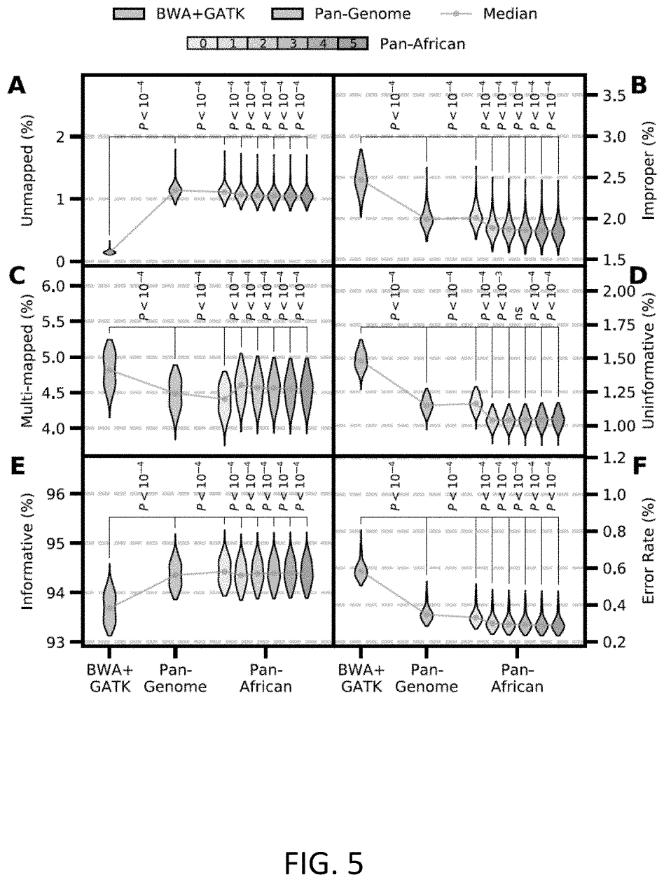

The pan-genome and population-specific graph references were compared with BWA-MEM for alignment and GATK for variant calling. First, the alignment accuracy was compared, as shown in FIG. 5. Each panel shows a different alignment statistic as a violin plot. Each violin corresponds to a different graph reference, representing the median and the distribution of the statistic over all benchmarking samples. Panel (a) shows the percentage of unmapped reads. BWA maps more reads compared to any of the graph references. This is due to the lenient alignment approach used by BWA as opposed to the more stringent criteria used by the graph aligner. It is seen that improper read (classified as either an improper orientation for read pairs or an insertion length outside the expected range) and uninformative read (MAPQ<20) percentages are much lower for graph approaches compared to BWA. The multi-mapped read ratio is also higher for BWA compared to any graph approach. As is readily seen from this example, using a graph reference construct generated using the techniques developed by the inventors and described herein results in improvements in read alignment over conventional techniques. By excluding variants that may introduce ambiguity into the graph reference construct (e.g., by excluding one or more structural variants and/or multiply-alignable variants), fewer reads align to multiple locations in the graph reference construct as compared to the conventional techniques, resulting in more accurate and reliable alignment results.

A useful metric to measure the representativeness of a population-specific graph is the alignment error rate, i.e., per-base mismatch rate with respect to the genome reference. A smaller error rate indicates that the genetic composition of the population is more successfully captured and also the reference bias is reduced. Panel (f) of FIG. 5 shows that the error rate consistently decreases from the linear approach to the Pan-African graphs. Each augmentation of the Pan-African graph achieves a better the error rate, leading to around 50% reduction compared to BWA in the last iteration.

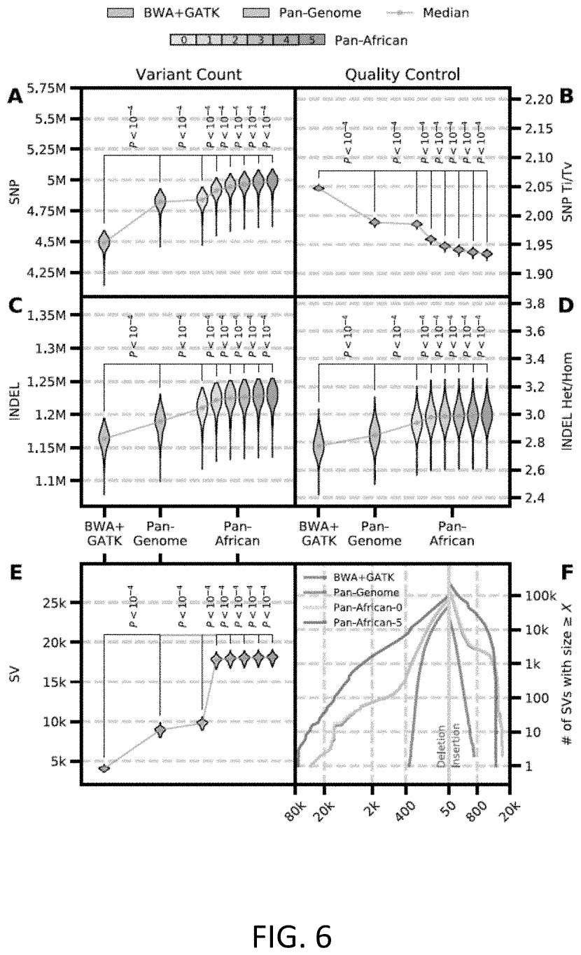

The utility of population-specific graphs for variant calling was also measured. A graph-aware variant caller that can make use of the information stored in graph references was used for variant calling. The overall performance on single nucleotide polymorphisms (SNPs), insertions and deletions (INDELs) and structural variants (SVs) for all graph references is shown in FIG. 6. Panels (a) and (c) show the number of SNPs and INDELs discovered per sample, respectively. It is seen that the Pan-Genome graph provides a higher sensitivity compared to the BWA+GATK pipeline. Therefore, using the graph reference construct generated using the techniques developed by the inventors and described herein, allows for improvements in variant calling over conventional techniques.

Panel (e) of FIG. 6 shows the number of SVs detected by each pipeline (SVs are defined as variants longer than 50 base pairs). The size distribution of SVs is also shown in Panel (f) for BWA+GATK, Pan-Genome, Pan-African 0 and Pan-African 5 pipelines. It is seen that the linear approach of using BWA+GATK has a significantly lower SV detection rate and can only detect short SVs. The Pan-Genome graph provides considerable improvement over the linear approach. This is made possible by the addition of the alt-contigs in the GRCh38 assembly as alternate paths into the graph reference. Therefore, using a graph reference construct, generated using the techniques developed by the inventors and described herein, allows for more accurate variant calling. By merging variants from different sources and including the merged variants in the graph reference construct, the resulting graph reference construct can be used for more accurate variant calling.

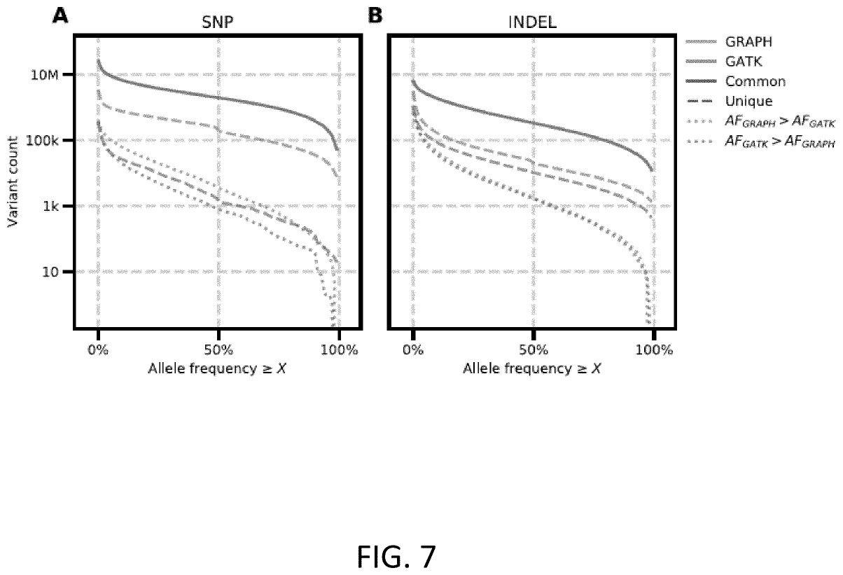

Using the output of the last iteration as the final graph reference, the variant calls made by the Pan-African 5 pipeline and those made by the BWA+GATK pipeline are compared in more detail. FIG. 7 shows the cumulative variant counts for both pipelines with respect to the allele frequency. The variants are first classified into SNPs and INDELs (panels A and B, respectively), and then into common (detected in the population by both pipelines) and unique (detected by either pipeline) variant sets. High concordance is observed between the pipelines as majority of the variants are detected by both pipelines (solid lines). In order to distinguish between the genotyping efficacy of these methods, common variants are further split into two categories as AF_{GRAF}>AF_{GATK} and AF_{GATK}>AF_{GRAF} (dotted lines). The former represents the number of variants that are detected in the population by both methods but genotyped with a higher sensitivity by the graph pipeline (and vice versa for the latter). Among the variants observed in the population with a high frequency (≅5%), graph pipeline is able to genotype approximately 120 k INDELs and 119 k SNPs with a higher AF, whereas the same numbers for GATK is 106 k INDELs and 51 k SNPs. Additionally, it is noteworthy that the graph-based approach identifies approximately 6 times as many unique variants as the linear method.

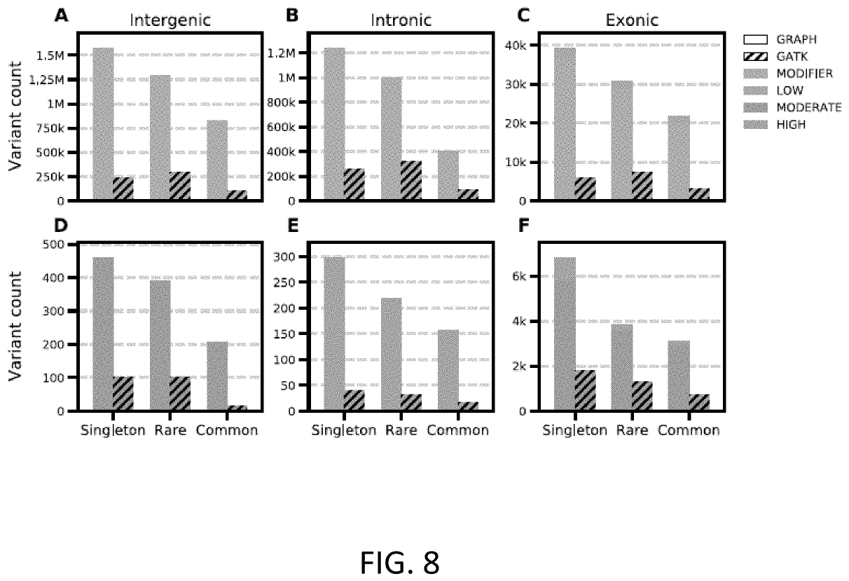

In order to predict the potential clinical significance of the variants detected by the graph-based approach and rule out any bias in variant calling sensitivity towards specific genomic regions or prevalence in population, all detected variants were stratified into exonic, intronic and intergenic regions, as shown in FIG. 8. The variants were further divided into three frequency bins as singleton (observed in only one sample), rare (AF<5% but observed in multiple samples) and common (AF≥5%), and the results were compared to the linear approach BWA+GATK. It is seen that the use of Pan-African graph leads to the detection of 3 to 4 times more high and moderate impact variants in exonic regions for all frequency bins, compared to the BWA+GATK pipeline (panel F). Specifically, there are 429 and 9457 more high and moderate impact variants, respectively, detected by the graph pipeline. It is seen that the use of Pan-African graph leads to the detection of 3 to 4 times more high and moderate impact variants in exonic regions for all frequency bins, compared to the BWA+GATK pipeline (panel F). Specifically, there are 429 and 9457 more high and moderate impact variants, respectively, detected by the graph pipeline. As is clear from this example, using a graph reference construct, generated using the techniques developed by the inventors and described herein, allows for increased sensitivity in variant calling over conventional techniques. The increased sensitivity allows for the detection of more high and moderate impact variants that can be used to predict the clinical significance of the detected variants.

### Additional Implementation Details

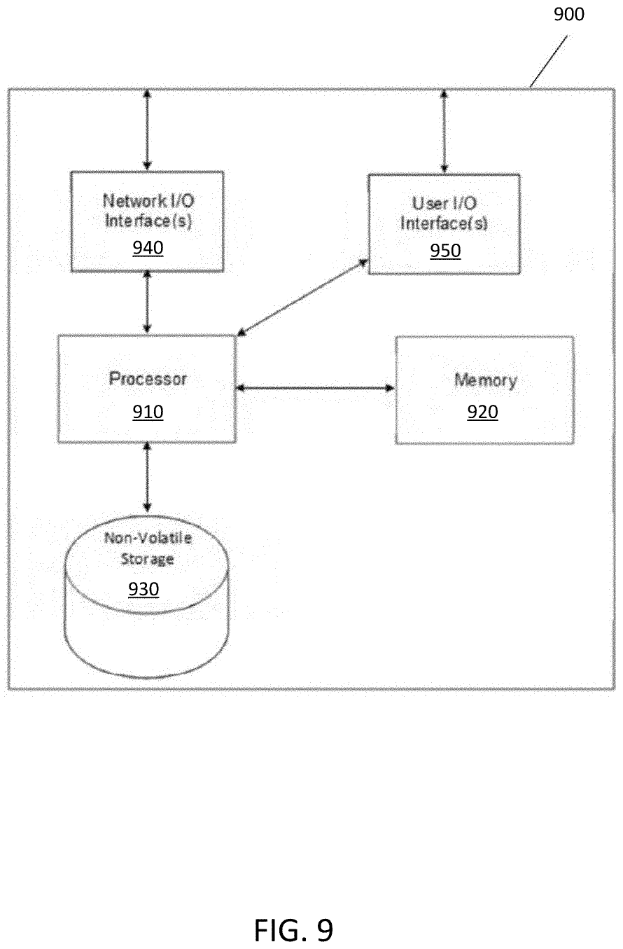

An illustrative implementation of a computer system 900 that may be used in connection with any of the embodiments of the technology described herein (e.g., such as the processes described with reference to FIGS. 2A-D and 4A-4D) is shown in FIG. 9. The computer system 900 includes one or more computer hardware processors 910 and one or more articles of manufacture that comprise non-transitory computer-readable storage media (e.g., memory 920 and one or more non-volatile storage media 930). The processor 910 may control writing data to and reading data from the memory 920 and the non-volatile storage device 930 in any suitable manner, as the aspects of the technology described herein are not limited in this respect. To perform any of the functionality described herein, the processor(s) 910 may execute one or more processor-executable instructions stored in one or more non-transitory computer-readable storage media (e.g., the memory 920), which may serve as non-transitory computer-readable storage media storing processor-executable instructions for execution by the processor 910.

Computing device 900 may also include a network input/output (I/O) interface 940 via which the computing device may communicate with other computing devices (e.g., over a network), and may also include one or more user I/P interfaces 950, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments may be implemented in any of numerous ways. For example, the embodiments may be implemented using hardware, software, or a combination thereof. When implemented in software, the software code can be executed on any suitable computer hardware processor (e.g., one or more microprocessors, one or more graphic processing units (GPUs)) or collection of computer hardware processors, whether provided in a single computing device or distributed among multiple computing devices. Additionally or alternatively, the embodiments may be implemented using one or more application specific integrated circuits (ASICs), and/or one or more field programmable gate arrays (FPGAs). As such, embodiments may be implemented using any suitable computing device (e.g., one or more computer hardware processors, one or more ASICs, and/or one or more FPGAs).

In this respect, it should be appreciated that one implementation of the embodiments described herein comprises at least one non-transitory computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (e.g., a plurality of executable instructions) that, when executed on one or more computer hardware processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for generating a graph reference construct, the method comprising:
using at least one computing device to perform:
obtaining a plurality of variants associated with a reference sequence construct for at least a portion of a genome; and
generating the graph reference construct using the plurality of variants and the reference sequence construct, the generating comprising:
filtering the plurality of variants to obtain a filtered set of variants, the filtered set of variants being a subset of the plurality of variants, the filtering comprising a plurality of filtering stages including a first filtering stage and a second filtering stage different from and performed subsequent to the first filtering stage,
the first filtering stage comprising identifying a first subset of variants from among the plurality of variants at least in part by excluding one or more structural variants from the plurality of variants, the one or more structural variants including a first structural variant;
the second filtering stage comprising identifying the filtered set of variants from among the first subset of variants at least in part by excluding one or more multiply-alignable variants from the first subset of variants;
generating the graph reference construct using the filtered set of variants and the reference sequence construct; and
outputting the generated graph reference construct.

2. The method of claim 1, wherein identifying the first subset of variants from among the plurality of variants comprises:
determining whether a first length of the first structural variant exceeds a first specified threshold; and
upon determining that the first length exceeds the first specified threshold, excluding the first structural variant from the plurality of variants.

3. The method of claim 2,
wherein the first structural variant is an insertion event, and
wherein determining whether the first length of the first structural variant exceeds the first specified threshold comprises determining whether the first length is at least 5,000 base pairs.

4. The method of claim 2,
wherein the first structural variant is a deletion event, and
wherein determining whether the first length of the first structural variant exceeds the first specified threshold comprises determining whether the first length is at least 90,000 base pairs.

5. The method of claim 1, wherein identifying the first subset of variants from among the plurality of variants comprises:
aligning the first structural variant to the reference sequence construct.

6. The method of claim 1, wherein identifying the first subset of variants from among the plurality of variants comprises:
determining whether the reference sequence construct includes a subsequence, wherein the subsequence is identical to at least a portion of the first structural variant; and
upon determining that the reference sequence construct includes the subsequence, excluding the first structural variant from the plurality of variants.

7. The method of claim 1, wherein identifying the first subset of variants from among the plurality of variants comprises:
aligning the first structural variant to one or more variants of the plurality of variants, the one or more variants being different from the first structural variant.

8. The method of claim 1, wherein identifying the first subset of variants from among the plurality of variants comprises:
determining whether a second structural variant includes a subsequence, wherein the subsequence is identical to at least a portion of the first structural variant; and
upon determining that the second structural variant includes the subsequence, excluding one of the first structural variant or the second structural variant from the plurality of variants.

9. The method of claim 1, wherein identifying the first subset of variants from among the plurality of variants comprises:
aligning the first structural variant to a decoy sequence associated with the reference sequence construct.

10. The method of claim 1, wherein identifying a first subset of variants from among the plurality of variants comprises:
determining whether a decoy sequence associated with the reference sequence construct includes a subsequence, wherein the subsequence is identical to at least a portion of the first structural variant; and
upon determining that the decoy sequence includes the subsequence, masking the decoy sequence.

11. The method of claim 1, wherein identifying the first subset of variants from among the plurality of variants further comprises, upon determining that the first length does not exceed the first specified threshold:
determining whether the reference sequence construct includes a first subsequence, wherein the first subsequence is identical to at least a first portion of the first structural variant; and
upon determining that the reference sequence construct includes the first subsequence, excluding the first structural variant from the plurality of variants.

12. The method of claim 11, wherein determining whether the reference sequence construct includes the first subsequence comprises determining whether the first subsequence has a length that is greater than a second specified threshold.

13. The method of claim 11, further comprising:
upon determining that the reference sequence construct does not include the first subsequence, determining whether a second structural variant includes a second subsequence, wherein the second subsequence is identical to at least a second portion of the first structural variant; and
upon determining that the second structural variant includes the second subsequence, excluding one of the first structural variant or the second structural variant from the plurality of variants.

14. The method of claim 13, wherein determining whether the second structural variant includes the second subsequence comprises determining whether the second subsequence has a length that is greater than the second specified threshold.

15. The method of claim 14, wherein the second specified threshold is at least 150 base pairs.

16. The method of claim 13, wherein excluding one of the first structural variant or the second structural variant from the plurality of variants comprises:
identifying a shortest variant from among the first structural variant and the second structural variant; and
excluding the shortest variant from the plurality of variants.

17. The method of claim 13, further comprising:
upon determining that the second structural variant does not include the second subsequence, determining whether a decoy sequence associated with the reference sequence construct includes a third subsequence, wherein the third subsequence is identical to at least a third portion of the first structural variant; and
upon determining that the decoy sequence includes the third subsequence, masking the decoy sequence.

18. The method of claim 1, wherein identifying the filtered set of variants from among the first subset of variants comprises:
generating an initial graph reference construct using at least some of the first subset of variants.

19. The method of claim 18, wherein identifying the filtered set of variants from among the first subset of variants further comprises:
generating a plurality of graph reads using the initial graph reference construct, wherein each of at least some of the plurality of graph reads are associated with a respective path in the initial graph reference construct.

20. The method of claim 19, wherein the plurality of graph reads comprise a first subset of graph reads and a second subset of graph reads, and wherein generating the plurality of graph reads comprises:
generating the first subset of graph reads by traversing the initial graph reference construct over a first interval; and
generating the second subset of graph reads by traversing the initial graph reference construct over a second interval, wherein the first interval and the second interval at least partially overlap.

21. The method of claim 19, wherein generating the plurality of graph reads comprises traversing the initial graph reference construct using a sliding window with a skip.

22. The method of claim 19, further comprising aligning at least some of the plurality of graph reads to the initial graph reference construct, the aligning comprising, for each graph read of the at least some of the plurality of graph reads:
determining a quality of alignment between the graph read and the graph reference construct; and
determining whether the quality of alignment exceeds a threshold.

23. The method of claim 22, further comprising identifying a first group of the at least some of the plurality of graph reads, wherein each graph read included in the first group of the at least some of the plurality of graph reads includes a first combination of one or more variants of the first subset of variants.

24. The method of claim 23, wherein the first group of the at least some of the plurality of graph reads includes a first graph read and a second graph read; and further comprising:
upon determining that neither a first quality of alignment determined for the first graph read nor a second quality of alignment determined for the second graph read exceed the specified threshold, excluding at least one multiply-alignable variant from the filtered set of variants.

25. The method of claim 24, wherein the at least one multiply-alignable variant is included in the first combination of the one or more variants.

26. The method of claim 1, wherein identifying the filtered set of variants from among the first subset of variants comprises:
generating an initial graph reference construct using the first subset of variants;
traversing the initial graph reference construct to generate a plurality of graph reads;
aligning the plurality of graph reads to the initial graph reference construct to determine qualities of alignment for each of at least some of the plurality of graph reads; and
excluding at least some of the one or more of the first set variants from the second set of variants based on the qualities of alignment.

27. The method of claim 26, wherein one or more of the plurality of graph reads are associated with a same combination of one or more of the first subset of variants; and
further comprising:
determining whether each of the qualities of alignment determined for the one or more of the plurality of graph reads is below a specified threshold; and
upon determining that each of the qualities of alignment is below the specified threshold, excluding at least one variant from the filtered set of variants.

28. The method of claim 1, wherein obtaining the plurality of variants comprises:
obtaining a plurality of alternative sequences associated with the reference sequence construct;
processing at least some of the plurality of alternative sequences, the processing comprising, for a first alternative sequence of the plurality of alternative sequences:
aligning the first alternative sequence to the reference sequence construct to obtain an aligned position;
identifying one or more differences between the first alternative sequence and the reference sequence construct at the aligned position; and
including at least some of the one or more differences as first variants in the plurality of variants.

29. The method of claim 28, further comprising, after processing the at least some of the plurality of alternative sequences, constructing an updated reference sequence construct that does not include the plurality of alternative sequences.

30. The method of claim 28, wherein the first alternative sequence includes an inverted sequence patch; and
wherein aligning the first alternative sequence to the reference sequence construct to obtain the aligned position comprises obtaining an alternative aligned position for the inverted sequence patch.

31. The method of claim 28, further comprising left normalizing the first variants with respect to the reference sequence construct before including the first variants in the plurality of variants.

32. The method of claim 28, wherein the at least some of the one or more differences include consecutive first and second differences, wherein the first difference is associated with a first subsequence of the first alternative sequence, and wherein the second difference is associated with a second subsequence of the reference sequence construct; and
further comprising processing the first and second differences before including them as first variants in the plurality of variants, the processing comprising:
determining whether the first subsequence includes one or more regions that are included in the second subsequence; and
upon determining that the first subsequence includes the one or more regions that are included in the second subsequence, removing the one or more regions from both the first and second subsequences.

33. The method of claim 32, wherein the first and second differences respectively comprise insertion and deletion events.

34. The method of claim 28, wherein obtaining the plurality of variants further comprises:
obtaining second variants associated with the reference sequence construct; and
including the second variants in the plurality of variants.

35. The method of claim 34, further comprising annotating the second variants with information indicative of sources of the second variants.

36. The method of claim 34, wherein at least some of the first variants are associated respectively with first allele frequencies and at least some of the second variants are associated respectively with second allele frequencies; and
further comprising, for a shared variant included in both the at least some of the first variants and the at least some of the second variants, averaging the first and second allele frequencies associated with the shared variant to obtain an averaged allele frequency.

37. A system, comprising:
at least one computer hardware processor; and
at least one non-transitory computer-readable storage medium storing processor-executable instructions that, when executed by the at least one computer hardware processor, cause the at least one computer hardware processor to perform:
obtaining a plurality of variants associated with a reference sequence construct for at least a portion of a genome;
generating the graph reference construct using the plurality of variants and the reference sequence construct, the generating comprising:
filtering the plurality of variants to obtain a filtered set of variants, the filtered set of variants being a subset of the plurality of variants, the filtering comprising a plurality of filtering stages including a first filtering stage and a second filtering stage different from and performed subsequent to the first filtering stage,
the first filtering stage comprising identifying a first subset of variants from among the plurality of variants at least in part by excluding one or more structural variants from the plurality of variants, the one or more structural variants including a first structural variant;
the second filtering stage comprising identifying the filtered set of variants from among the first subset of variants at least in part by excluding one or more multiply-alignable variants from the first set of variants; and
generating the graph reference construct using the filtered set of variants and the reference sequence construct; and
outputting the generated graph reference construct.

38. At least one non-transitory computer-readable storage medium storing processor-executable instructions that, when executed by at least one computer hardware processor, cause the at least one computer hardware processor to perform:
obtaining a plurality of variants associated with a reference sequence construct for at least a portion of a genome;
generating the graph reference construct using the plurality of variants and the reference sequence construct, the generating comprising:
filtering the plurality of variants to obtain a filtered set of variants, the filtered set of variants being a subset of the plurality of variants, the filtering comprising a plurality of filtering stages including a first filtering stage and a second filtering stage different from and performed subsequent to the first filtering stage,
the first filtering stage comprising identifying a first subset of variants from among the plurality of variants at least in part by excluding one or more structural variants from the plurality of variants, the one or more structural variants including a first structural variant;
the second filtering stage comprising identifying the filtered set of variants from among the first subset of variants at least in part by excluding one or more multiply-alignable variants from the first set of variants; and
generating the graph reference construct using the filtered set of variants and the reference sequence construct; and
outputting the generated graph reference construct.
